(19) United States
(12) Patent Application Publication
(10) Pub. No.: US 2010/0075297 A1
(43) Pub. Date: Mar. 25, 2010

(54) PHOTOSENSITIVE POLYPEPTIDE AND METHODS OF THEIR PRODUCTION AND USE

(76) Inventor: David S. Lawrence, Chapel Hill, NC (US)

Correspondence Address: AMSTER, ROTHSTEIN & EBENSTEIN LLP, 90 PARK AVENUE, NEW YORK, NY, 10016, US

(21) Appl. No.: 12/448,097

(22) PCT Filed: Dec. 13, 2007
(86) PCT No.: PCT/US07/25468

§ 371 Date: Dec. 2, 2009

Related U.S. Application Data
(60) Provisional application No. 60/876,297, filed on Dec. 20, 2006.

(51) Int. Cl. C12Q 1/00; C07K 1/00; C07K 2/00; C07K 5/08; C07K 5/10; G01N 33/53; C07K 7/06

(52) U.S. Cl. 435/4; 530/333; 530/300; 530/331; 530/330; 436/501; 530/328; 530/329

(57) ABSTRACT

Methods for producing polypeptides caged with a photolabile group on one or more backbone nitrogens are provided, in which the photolabile group is added to a growing polypeptide chain during polypeptide synthesis. Photosensitive polypeptides produced by the methods are described, as are methods of using such photosensitive polypeptides to assay enzyme activity or inhibit protein-protein interactions. Methods for producing polypeptides caged with a photolabile group on one or more side chain nitrogens, where the photolabile group is incorporated into the side chain during polypeptide synthesis, are also provided.

### CROSS-REFERENCE TO RELATED APPLICATIONS

This application claims the benefit of U.S. Provisional Patent Application No. 60/876,297, filed on Dec. 20, 2006, the content of which is hereby incorporated by reference.

### STATEMENT OF GOVERNMENT SUPPORT

This invention was made with United States government support under Grant Nos. GM067198 and CA79954 from the National Institutes of Health. Accordingly, the United States government has certain rights in this invention.

### FIELD OF THE INVENTION

The present invention relates to synthesis of photosensitive polypeptides, in particular, polypeptides having a photolabile group on a backbone or side chain nitrogen, where the photolabile group is introduced during synthesis of the polypeptide. Compositions, kits, and systems that include such photosensitive polypeptides are also provided, as are methods of using such photosensitive polypeptides to assay enzyme activity or control timing of inhibition of protein-protein interactions.

### BACKGROUND OF THE INVENTION

Living cells have been referred to as the test tubes of the 21^{st} century (Hansen and Oddershede (2005) Proc. SPIE 5930:593003-1-9). However, although a host of reagents have been described for inhibiting, manipulating, or visualizing a wide variety of intracellularly-relevant processes and several promising strategies have been reported for the delivery of otherwise impermeable reagents across the cell membrane barrier (see, e.g., Gupta et al. (2005) Adv. Drug Deliv. Rev. 57:637-51 and Morris (2006) Biochim. Biophys. Acta 1758:384-93), key challenges remain before the cell-as-a-test-tube analogy can be fully realized. For example, a particularly confounding attribute that differentiates living cell biochemistry from its counterpart in the test tube is that the cell, not the investigator, controls where and when a given transformation occurs.

Among other benefits, the present invention provides light-activatable (photosensitive or caged) compounds that overcome the above noted difficulty by allowing the investigator to retain control over the activity of bio-reagents, even after they have entered the cell. A complete understanding of the invention will be obtained upon review of the following.

### SUMMARY OF THE INVENTION

One aspect of the invention provides techniques for synthesizing photosensitive polypeptides by introducing a photolabile group onto a backbone nitrogen during synthesis of the polypeptide. Thus, one general class of embodiments provides methods of making a polypeptide comprising a photolabile group covalently bonded to a backbone nitrogen of a first residue. In the methods, during chemical synthesis of the polypeptide (e.g., liquid-phase or solid-phase synthesis), the first residue is incorporated into a growing polypeptide to produce an incorporated first residue. An N-terminal amine of the incorporated first residue is then reacted with at least a first caging compound, to covalently bond the photolabile group to the backbone nitrogen of the first incorporated residue.

In a related aspect, the invention provides techniques for synthesizing photosensitive polypeptides by introducing a photolabile group onto a side chain nitrogen during synthesis of the polypeptide. Thus, another general class of embodiments provides methods of making a polypeptide comprising a photolabile group covalently bonded to a side chain nitrogen of a first residue. In the methods, during chemical synthesis of the polypeptide, the first residue is incorporated into a growing polypeptide to produce an incorporated first residue. A side chain amine of the incorporated first residue is then reacted with at least a first caging compound, to covalently bond the photolabile group to the side chain nitrogen of the first incorporated residue.

The invention also features photosensitive polypeptides produced by the methods, as well as kits including and methods employing such polypeptides. Thus, for example, one class of embodiments provides a composition that includes a photosensitive polypeptide, which photosensitive polypeptide comprises a polypeptide substrate for an enzyme and at least one photolabile group covalently bonded to a backbone nitrogen of the polypeptide substrate, which photolabile group inhibits the enzyme from acting on the substrate. The composition optionally also includes the enzyme.

Another class of embodiments provides methods of assaying an activity of an enzyme. In the methods, the enzyme and a photosensitive polypeptide are contacted. The photosensitive polypeptide comprises a polypeptide substrate for the enzyme and at least one photolabile group covalently bonded to a backbone nitrogen of the polypeptide substrate, which photolabile group inhibits (e.g., prevents) the enzyme from acting on the substrate. The assay is initiated by exposing the enzyme and the photosensitive polypeptide to light of a first wavelength, thereby removing the photolabile group from the polypeptide substrate, and the activity of the enzyme is assayed.

Yet another class of embodiments provides a kit for detecting an activity of an enzyme. The kit includes a photosensitive polypeptide and instructions for using the photosensitive polypeptide to detect activity of the enzyme, packaged in one or more containers. The photosensitive polypeptide comprises a polypeptide substrate for the enzyme, a label, wherein a signal from the label is sensitive to the state of the substrate, and at least one photolabile group covalently bonded to a backbone nitrogen of the polypeptide substrate, which photolabile group inhibits (e.g., prevents) the enzyme from acting on the substrate.

Another class of embodiments provides a composition that includes a photosensitive polypeptide, which photosensitive polypeptide comprises an inhibitory polypeptide that competes with a first polypeptide for binding to a second polypeptide and at least one photolabile group covalently bonded to a backbone nitrogen of the inhibitory polypeptide. The photolabile group inhibits (e.g., prevents) the inhibitory polypeptide from binding to the second polypeptide. The composition optionally also includes the first polypeptide and/or the second polypeptide.

Another class of embodiments provides methods of inhibiting interaction between a first polypeptide and a second polypeptide. In the methods, a photosensitive inhibitory polypeptide is provided that comprises an inhibitory polypeptide that competes with the first polypeptide for binding to the second polypeptide and at least one photolabile group covalently bonded to a backbone nitrogen of the inhibitory polypeptide, which photolabile group inhibits (e.g., prevents) the inhibitory polypeptide from binding to the second polypeptide. The photosensitive inhibitory polypeptide, the first polypeptide, and the second polypeptide are contacted, and the photolabile group is removed from the photosensitive inhibitory polypeptide by exposing the photosensitive inhibitory polypeptide to light of a first wavelength, thereby permitting the inhibitory polypeptide to bind to the second polypeptide in competition with the first polypeptide.

Yet another class of embodiments provides a kit for inhibiting interaction between a first polypeptide and a second polypeptide. The kit includes a photosensitive inhibitory polypeptide and instructions for using the photosensitive inhibitory polypeptide to inhibit binding of the first polypeptide to the second polypeptide, packaged in one or more containers. The photosensitive inhibitory polypeptide comprises an inhibitory polypeptide that competes with the first polypeptide for binding to the second polypeptide and at least one photolabile group covalently bonded to a backbone nitrogen of the inhibitory polypeptide, which photolabile group inhibits (e.g., prevents) the inhibitory polypeptide from binding to the second polypeptide.

One class of embodiments provides a composition that includes a photosensitive polypeptide, which photosensitive polypeptide comprises a first residue comprising a secondary amine in which a photolabile group is covalently bonded to a side chain nitrogen.

### BRIEF DESCRIPTION OF THE DRAWINGS

FIG. 1 schematically illustrates synthesis of photosensitive polypeptide 4. The structures of corresponding analogue 5 and of parent peptide 6 are also depicted.

FIG. 2 depicts the structures of coumarin derivative 7, resin 8, chymotrypsin substrate 9 and its photosensitive caged analogue 10, and coumarin derivative B.

FIG. 3 depicts a graph illustrating that caged peptide 10 (curve a) is not hydrolyzed by chymotrypsin as assessed by a continuous assay. Photolysis times of 5 (curve b), 10 (curve c), 15 (curve d), and 20 (curve e) minutes produce increasing amounts of active substrate and therefore increasing reaction rates and product.

FIG. 4 depicts the structure of photosensitive PKA substrate 12.

FIG. 5 depicts a graph illustrating that caged peptide 12 (◯) is not phosphorylated by PKA as assessed by a fixed time point assay. Photolysis times of 5 (◯), 10 (Δ), 15 (⋄), and 20 (X) minutes produce increasing amounts of active substrate and therefore increasing reaction rates and product.

FIG. 6 schematically illustrates operation of SH2 sensor peptide A, which exhibits an approximately 10-fold enhanced fluorescence upon binding to the Lck SH2 domain.

FIG. 7 schematically illustrates the competition assay used to assess the K_{d} values of peptides 4-6.

FIG. 8 depicts a graph of fluorescence change as a function of photolysis time of caged peptide 4, as measured in the competition assay of FIG. 7.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, reference to “a molecule” includes a plurality of molecules, and the like.

The term “about” as used herein indicates the value of a given quantity varies by +/−10% of the value, or optionally +/−5% of the value, or in some embodiments, by +/−1% of the value so described.

An “amino acid sequence” is a polymer of amino acid residues (a protein, polypeptide, etc.) or a character string representing an amino acid polymer, depending on context.

A “caging compound” (or “caging reagent”) refers to a compound that is reacted with a polypeptide to introduce a photolabile group onto the polypeptide.

A “Dab residue” is a (L)-2,4-diaminobutyric acid residue.

A “Dap residue” is a (L)-2,3-diaminopropionic acid residue.

An “enzyme” is a biological macromolecule that has at least one catalytic activity (i.e., that catalyzes at least one chemical reaction). An enzyme is typically a protein, but can be, e.g., RNA. Known protein enzymes have been grouped into six classes (and a number of subclasses and sub-subclasses) under the Enzyme Commission classification scheme (see, e.g. the Nomenclature Committee of the International Union of Biochemistry and Molecular Biology enzyme nomenclature pages, on the world wide web at www (dot) chem (dot) qmul (dot) ac (dot) uk/iubmb/enzyme), namely, oxidoreductase, transferase, hydrolase, lyase, ligase, or isomerase. The activity of an enzyme can be “assayed,” either qualitatively (e.g., to determine if the activity is present) or quantitatively (e.g., to determine how much activity is present or kinetic and/or thermodynamic constants of the reaction).

A “kinase” is an enzyme that catalyzes the transfer of a phosphoryl group from one molecule to another. A “protein kinase” is a kinase that transfers a phosphoryl group to a protein, typically from a nucleotide such as ATP. A “tyrosine protein kinase” (or “tyrosine kinase”) transfers the phosphoryl group to a tyrosine side chain (e.g., a particular tyrosine), while a “serine/threonine protein kinase” (“serine/threonine kinase”) transfers the phosphoryl group to a serine or threonine side chain (e.g., a particular serine or threonine).

A “label” is a moiety that facilitates detection of a molecule. Common labels in the context of the present invention include fluorescent, luminescent, and/or colorimetric labels. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

An “environmentally sensitive label” is a label whose signal changes when the environment of the label changes. For example, the fluorescence of an environmentally sensitive fluorescent label changes when the hydrophobicity, pH, and/or the like of the label's environment changes (e.g., upon binding of the molecule with which the label is associated to another molecule such that the label is transferred from an aqueous environment to a more hydrophobic environment at the molecular interface).

A “phosphatase” is an enzyme that removes a phosphate group from a molecule. A “protein phosphatase” removes the phosphate group from an amino acid side chain in a protein. A “serine/threonine-specific protein phosphatase” removes the phosphate from a serine or threonine side chain (e.g., a particular serine or threonine), while a “tyrosine-specific protein phosphatase” removes the phosphate from a tyrosine side chain (e.g., a particular tyrosine).

A “photolabile group” is a moiety whose covalent attachment to a molecule (e.g., a polypeptide) is reversed (cleaved) by exposure to light of an appropriate wavelength.

A “photosensitive polypeptide” is a polypeptide that comprises at least one photolabile group. In embodiments in which the photolabile group blocks, inhibits, or interferes with an activity (e.g., the biological activity) of the polypeptide, removal of the photolabile group by exposure to light of an appropriate wavelength restores the activity of the polypeptide.

A “polypeptide” is a polymer comprising two or more amino acid residues (e.g., a peptide or a protein). The polymer can additionally comprise non-amino acid elements such as labels, blocking groups, or the like and can optionally comprise modifications such as glycosylation or the like. The amino acid residues of the polypeptide can be natural or non-natural and can be unsubstituted, unmodified, substituted or modified. A “backbone nitrogen” is a nitrogen atom which is part of the polypeptide's backbone (also called the main chain), while a “side chain nitrogen” is a nitrogen atom which is part of one of the polypeptide's side chains.

A “synthetic polypeptide” is a polypeptide made through in vitro chemical synthesis, as opposed to a polypeptide made either in vitro or in vivo by a template-directed, enzyme-dependent reaction. Synthetic polypeptides optionally include fewer than 100 residues, for example, fewer than 75 residues or fewer than 50 residues.

The “N-terminal amine” of a given polypeptide refers to the amine moiety of that polypeptide's N-terminal residue.

A “quencher” is a moiety that alters a property of a label (typically, a fluorescent label) when it is in proximity to the label. For example, the quencher can quench (reduce the intensity of) a fluorescent emission from a fluorescent label when it is proximal to the label as compared to when not proximal to the label. A quencher can be, e.g., an acceptor fluorophore that operates via energy transfer and re-emits the transferred energy as light. Other similar quenchers, called “dark quenchers,” do not re-emit transferred energy via fluorescence.

A “subsequence” or “fragment” is any portion of an entire sequence, up to and including the complete sequence. Typically a subsequence or fragment comprises less than the full-length sequence.

A “substrate” is a molecule on which an enzyme acts. The substrate is typically supplied in a first state on which the enzyme acts, converting it to a second state. The second state of the substrate is then typically released from the enzyme.

A variety of additional terms are defined or otherwise characterized herein.

### DETAILED DESCRIPTION

Photosensitive polypeptides that can be activated by removal of photolabile groups upon exposure to light are useful tools for analysis of enzyme activity, protein-protein interactions, signaling pathways, and the like, both in vitro and in vivo. Use of photosensitive polypeptides allows initiation of such reactions to be tightly and conveniently controlled, temporally and/or spatially, since the polypeptides are optionally biologically inactive until the photolabile groups are removed. See, e.g., U.S. patent application publication no. 20040166553 by Nguyen et al. entitled “Caged sensors, regulators and compounds and uses thereof” and Lawrence (2005) “The preparation and in vivo applications of caged peptides and proteins” Cur. Opin. Chem. Biol. 9:570-5.

One strategy for production of photosensitive polypeptides is to modify the side chains with photolabile groups. See, e.g., U.S. Pat. No. 5,998,580 to Fay et al. entitled “Photosensitive caged macromolecules.” Another strategy is to attach a photolabile group to the polypeptide backbone, for example, to a backbone nitrogen. Such backbone caged polypeptides have been synthesized through addition of a photolabile group to the amino group of an amino acid followed by incorporation of the caged amino acid residue into a polypeptide (Tatsu et al. (2002) “A caged sperm-activating peptide that has a photocleavable protecting group on the backbone amide” FEBS Letters 525:20-24 and Johnson and Kent (2006) “Synthesis, stability and optimized photolytic cleavage of 4-methoxy-2-nitrobenzyl backbone-protected peptides” Chem Commun 1557-1559). This approach, however, requires synthesis of a different caged amino acid for each different type of residue to whose backbone nitrogen a photolabile group is to be attached. A related approach involves synthesis of the amino acid residue and simultaneous incorporation of the photolabile group during polypeptide synthesis (Johnson and Kent, supra), but this approach is applicable only to glycine. Synthesis of a photosensitive O-acyl isopeptide whose conversion to an N-acyl peptide is triggered by light has also been described (Taniguchi et al. (2006) “‘Click peptide’ based on the ‘O-acyl isopeptide method’: Control of Aβ1-42 production from a photo-triggered Aβ1-42 analogue” J Am Chem Soc 128:696-697). The isopeptide approach, however, is restricted to sequences with appropriately placed hydroxyamino residues (e.g., serine or threonine).

One aspect of the present invention provides methods for producing polypeptides caged with a photolabile group on one or more backbone nitrogens. Unlike in the approaches described above, in the methods of the invention, a photolabile group is added to the growing polypeptide chain during polypeptide synthesis. The N-terminal amine of the growing polypeptide is reacted with a caging compound to add the photolabile group to the growing polypeptide, permitting the photolabile group to be added at essentially any desired position in the polypeptide and rendering synthesis of various caged amino acids prior to polypeptide synthesis unnecessary.

Another aspect of the invention provides methods for caging side chains by introducing a photolabile group to a side chain nitrogen during polypeptide synthesis.

Photosensitive polypeptides produced by any of the methods are also described, as are methods of using such photosensitive polypeptides, e.g., to assay enzyme activity or inhibit protein-protein interactions.

### Chemical Synthesis of Polypeptides

Polypeptides can be synthesized by techniques that are known to those skilled in the peptide art. Descriptions of the many techniques available are found, e.g., in Merrifield (1963) J. Amer. Chem. Soc. 85:2149-2154; Stewart and Young (1984) Solid Phase Peptide Synthesis, 2nd ed., J. D. Pierce Chemical Company, Rockford, Ill.; Atherton et al. (1989) Solid Phase Peptide Synthesis: A Practical Approach, IRL Press; Fields et al. (1992) Synthetic Peptides: A User's Guide, Grant, G. A., ed., W. H. Freeman and Co., New York; Benoiton (2005) Chemistry of Peptide Synthesis, CRC; and Chan and White, eds. (2000) Fmoc Solid Phase Peptide Synthesis: A Practical Approach, Oxford University Press, USA. See also the Examples sections herein below.

Polypeptides can be synthesized by liquid-phase or, more typically, by solid-phase peptide synthesis techniques. In general, in solid-phase peptide synthesis, synthesis proceeds from the C-terminus to the N-terminus. The C-terminal residue is attached to a solid support (e.g., a resin such as a polystyrene or polyamide resin) on which the polypeptide is constructed by sequential addition of residues. The N-terminal amine of each amino acid monomer is protected by groups such as Boc (tert-butoxycarbonyl) or Fmoc (9-fluorenylmethyl carbamate), and the protected monomer is added onto a deprotected amino acid chain.

The basic Fmoc method, for example, involves performing repetitive cycles of coupling the activated C-terminus of an Fmoc-amino acid to the N-terminus of the growing resin-linked peptide chain. The Fmoc protecting group is then removed from the newly incorporated residue under basic conditions, and the cycle is repeated with the next Fmoc-amino acid. Reactive side chains are blocked with stable protecting groups, e.g., t-butyl ether (used to block Ser, Thr, and Tyr), t-butyl ester (used to block Asp and Glu), trityl (used to block His, Cys, Asn and Gln), or butyloxycarbonyl (used to block Lys). At the completion of synthesis, protecting groups are removed as the peptide is cleaved from the resin (e.g., using a strong acid such as TFA). Upon cleavage, the polypeptide is recovered using standard methods, e.g., by using diethyl ether, and is optionally purified according to standard methods, e.g., reverse-phase HPLC. The identity of the synthesized polypeptide is optionally confirmed by conventional techniques such as microsequencing, NMR, amino acid analysis, and mass spectrometry.

The basic Boc method is similar, although acidic, rather than basic, conditions are used to remove Boc from the growing polypeptide prior to addition of the next Boc-amino acid. The Boc method is thus employed for synthesis of base-sensitive species; it can also be employed for complex syntheses.

Polypeptide synthesis can be automated using instruments commercially available, e.g., from Rainin Instrument Co. (Woburn, Mass.), Millipore Corp. (Milford, Mass.), Gilson Inc. (Middleton, Wis.), or Applied Biosystems (Foster City, Calif.).

### Synthesis of Backbone Caged Polypeptides

One aspect of the invention provides techniques for synthesizing photosensitive polypeptides by introducing a photolabile group onto a backbone nitrogen during synthesis of the polypeptide. Thus, one general class of embodiments provides methods of making a polypeptide comprising a photolabile group covalently bonded to a backbone nitrogen of a first residue. In the methods, during chemical synthesis of the polypeptide (e.g., liquid-phase or solid-phase synthesis), the first residue is incorporated into a growing polypeptide to produce an incorporated first residue. An N-terminal amine of the incorporated first residue is then reacted with at least a first caging compound, to covalently bond the photolabile group to the backbone nitrogen of the first incorporated residue. In embodiments in which the amine of the first residue was protected by Fmoc or Boc (or another, similar group) during incorporation of the first residue, the N-terminal amine is preferably deprotected prior to reaction with the first caging compound.

The first residue (i.e., the residue whose backbone nitrogen is being caged, which is not necessarily either the N-terminal residue of the final polypeptide product or the first residue to be incorporated into the polypeptide) can be an amino acid residue or a residue other than an amino acid residue (for example, a label such as a fluorophore, e.g., a coumarin derivative). Amino acid residues include the 20 standard α-L-amino acids used in in vivo protein synthesis, as well as other natural amino acids such as selenocysteine, selenomethionine, and pyrrolysine, other α-L-amino acids, D-amino acids, and β-amino acids. Amino acid residues include both natural and unnatural and standard and nonstandard residues. The precursor to the first residue that is incorporated can be essentially any molecule comprising an amine group and a carboxylic, sulfonic, or phosphonic acid group; thus, the residue can be essentially any moiety that can be incorporated into the polypeptide backbone via one amide, sulfonamide, or phosphonamide bond to one other residue or via two amide, sulfonamide, and/or phosphonamide bonds to two other residues.

In one class of embodiments, the N-terminal amine of the incorporated first residue is a primary amine. In these embodiments, reaction with the caging compound typically produces a secondary amine in which the photolabile group is bonded to the nitrogen. In other embodiments, the N-terminal amine of the incorporated first residue is a secondary amine other than an acylated amine (i.e., other than an amide).

The photolabile group can be placed at essentially any desired backbone nitrogen other than that of an internal proline residue. The first residue can thus be the N-terminal residue of the polypeptide, internal to the polypeptide, or the C-terminal residue of the polypeptide. The growing polypeptide (the polypeptide chain which is in the process of being synthesized and which, at least before the addition of the first residue, is not yet full length) optionally includes one or more residues, or the first residue can be the first residue incorporated into the polypeptide.

In embodiments in which the first residue is not the N-terminal residue of the final polypeptide product, the methods include, after the reacting step, incorporating at least a second residue N-terminal to the incorporated first residue. The second residue is typically a second amino acid residue, but it can be another type of residue. In embodiments in which addition of the caging group to the N-terminal amine of the first residue produces a secondary amine, incorporating a second amino acid (or other) residue N-terminal to the incorporated first amino acid residue can involve reacting a second amino acid or a protected form thereof with the secondary amine of the incorporated first amino acid residue. The reaction is typically performed in the presence of an activating agent such as bromo-tris-pyrrolidino phosphoniumhexafluorophosphate (PyBrop).

Steps of the method are optionally repeated to cage the backbone nitrogen of the second residue or of one or more other residues elsewhere in the polypeptide, if desired. Similarly, backbone caging is optionally employed in conjunction with side chain caging; one or more amino acid residues with caged side chains are optionally incorporated into the polypeptide, or a side chain nitrogen can be caged as described herein below. The methods optionally include incorporating a label and/or a quencher into the polypeptide, for example, as a residue or as part of an amino acid residue.

In one class of embodiments, the photolabile group is a derivative of a 2-nitrobenzyl group (also called an ortho-nitrobenzyl group). A derivative of a 2-nitrobenzyl group is or includes a substituted 2-nitrobenzyl moiety. For example, the photolabile group can have the structure

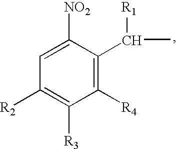

where, independently, R₁ is —H, —CH₃, —CONH₂, or —COO⁻ and R₂, R₃, and R₄ are independently —H, —CH₃, —OCH₃, —CH₂COO⁻, —OH, or —NO₂. The photolabile group is optionally a 4,5-dimethoxy-2-nitrobenzyl (DMNB) group, a 4-methoxy-2-nitrobenzyl group, a 2-nitrobenzyl group, a 2-nitrophenylethyl (NPE) group, or a 1-(4,5-dimethoxy-2-nitrophenyl)ethyl (DMNPE) group. As another example, the photolabile group can be a derivative of nitrobenzofuran. In one embodiment, the photolabile group is a nitrodibenzofuranyl group (NDBF; see Momotake et al. (2006) “The nitrodibenzofuran chromophore: a new caging group for ultra-efficient photolysis in living cells” Nat Methods 3:35-40), which has the structure

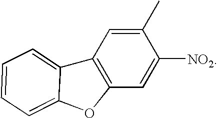

A number of compounds are known in the art that can be used as caging compounds in various types of reactions in the methods. For example, the N-terminal amine can be reductively alkylated with the first caging compound. Suitable first caging compounds for this approach include, but are not limited to, 4,5-dimethoxy-2-nitrobenzaldehyde, 4-methoxy-2-nitrobenzaldehyde, and 2-nitrobenzaldehyde. As another example, the N-terminal amine can be directly alkylated with a benzyl halide; exemplary first caging compounds for this approach include, but are not limited to, 4,5-dimethoxy-2-nitrobenzylbromide, 4-methoxy-2-nitrobenzylbromide, and 2-nitrobenzylbromide.

Polypeptides produced by the methods and compositions formed while practicing the methods are also features of the invention. Thus, for example, one class of embodiments provides a composition comprising a growing polypeptide (e.g., including one or more residues and attached to a resin or other solid support) with an unprotected N-terminal amine and at least a first caging compound. Another class of embodiments provides a composition including a polypeptide having a photolabile group attached to its N-terminal backbone nitrogen, an amino acid (e.g., a Boc- or Fmoc-amino acid), and optionally PyBrop or another activating agent. Other embodiments provide photosensitive synthetic polypeptides having photolabile groups covalently bonded to backbone nitrogens, as in the exemplary embodiments described below.

### Synthesis of Side Chain Caged Polypeptides

In a related aspect, the invention provides techniques for synthesizing photosensitive polypeptides by introducing a photolabile group onto a side chain nitrogen during synthesis of the polypeptide. Thus, one general class of embodiments provides methods of making a polypeptide comprising a photolabile group covalently bonded to a side chain nitrogen of a first residue. In the methods, during chemical synthesis of the polypeptide (e.g., liquid-phase or solid-phase synthesis), the first residue is incorporated into a growing polypeptide to produce an incorporated first residue. A side chain amine of the incorporated first residue is then reacted with at least a first caging compound, to covalently bond the photolabile group to the side chain nitrogen of the first incorporated residue.

The side chain amine of the first residue is optionally protected with a protecting group during incorporation of the first residue. This protecting group is then removed prior to reaction of the side chain amine of the first residue with the first caging compound. The protecting group is typically one that can be removed without removing protecting groups from other side chains in the growing polypeptide, such that only the desired, first side chain is available for reaction with the caging compound. For example, the side chain nitrogen can be protected with a group that can be removed under mildly acidic conditions that do not remove any other protecting groups present, such as a methyltrityl (MTT) or monomethoxytrityl (MMT) group.

As for the embodiments described above, the first residue (i.e., the residue whose side chain nitrogen is being caged, which is not necessarily either the N-terminal residue of the final polypeptide product or the first residue to be incorporated into the polypeptide) can be an amino acid residue (e.g., a natural, unnatural, standard or nonstandard residue) or a residue other than an amino acid residue, as long as the residue has a side chain nitrogen to which the photolabile group can be attached. For example, in one class of embodiments, the first residue is an amino acid residue selected from the group consisting of lysine, ornithine, (L)-2,3-diaminopropionic acid (Dap), (L)-2,4-diaminobutyric acid (Dab), homolysine, and aminophenylalanine.

In one class of embodiments, the side chain amine of the incorporated first residue is a primary amine. In these embodiments, reaction with the caging compound typically produces a secondary amine in which the photolabile group is bonded to the nitrogen. In other embodiments, the side chain amine of the incorporated first residue is a secondary amine.

The photolabile group can be attached to the side chain of a residue that occupies essentially any desired position in the polypeptide. The first residue can thus be the N-terminal residue of the polypeptide, internal to the polypeptide, or the C-terminal residue of the polypeptide.

In embodiments in which the first residue is not the N-terminal residue of the final polypeptide product, the methods include, after the reacting step, incorporating at least a second residue N-terminal to the incorporated first residue. Steps of the method are optionally repeated to cage a side chain nitrogen of the second residue or of one or more other residues elsewhere in the polypeptide, if desired. Similarly, side chain caging is optionally employed in conjunction with backbone caging or with another form of side chain caging. The methods optionally include incorporating a label and/or a quencher into the polypeptide.

Essentially all of the features noted for the methods above apply to these embodiments as well, as relevant: for example, with respect to type of photolabile group, caging compound, and/or the like. For example, in one class of embodiments, the photolabile group is a derivative of a 2-nitrobenzyl group, e.g., a 4,5-dimethoxy-2-nitrobenzyl group, a 4-methoxy-2-nitrobenzyl group, a 2-nitrobenzyl group, a 2-nitrophenylethyl group, a 1-(4,5-dimethoxy-2-nitrophenyl)ethyl group, or a nitrodibenzofuranyl group. Exemplary first caging compounds include, but are not limited to, 4,5-dimethoxy-2-nitrobenzaldehyde, 4-methoxy-2-nitrobenzaldehyde, 2-nitrobenzaldehyde, 4,5-dimethoxy-2-nitrobenzylbromide, 4-methoxy-2-nitrobenzylbromide, and 2-nitrobenzylbromide.

Polypeptides produced by the methods and compositions formed while practicing the methods are also features of the invention. Thus, for example, one class of embodiments provides a composition comprising at least a first caging compound and a growing polypeptide (e.g., including one or more residues and attached to a resin or other solid support), where the N-terminal residue of the growing (not necessarily complete) polypeptide has an unprotected amine group on the side chain. Other embodiments provide photosensitive synthetic polypeptides having photolabile groups covalently bonded to side chain nitrogens, as in the exemplary embodiments described below.

### Amide Caged Polypeptides

The methods of the invention, and the resulting photosensitive polypeptides, find use in a wide variety of applications. For example, for a photosensitive polypeptide in which the presence of the photolabile group interferes with biological activity of the polypeptide, when and where the polypeptide is active can be readily controlled by controlling when and where the photosensitive polypeptide is exposed to light to remove the photolabile group. Polypeptides whose activity can be controlled in this manner include, but are not limited to, enzyme substrates and polypeptides that interact with other polypeptides (e.g., ligands and inhibitors).

### Photosensitive Enzyme Substrates and Uses Thereof

One general class of embodiments provides a composition that includes a photosensitive polypeptide, which photosensitive polypeptide comprises a polypeptide substrate for an enzyme and at least one photolabile group covalently bonded to a backbone nitrogen of the polypeptide substrate, which photolabile group inhibits the enzyme from acting on the substrate.

The composition optionally also includes the enzyme. The enzyme can be essentially any enzyme that acts on a polypeptide substrate. For example, the enzyme can be a transferase, hydrolase, oxidoreductase, lyase, ligase, or isomerase. In one embodiment, the enzyme catalyzes a posttranslational modification of a polypeptide, for example, phosphorylation, acetylation, methylation, ubiquitination, sumoylation, glycosylation, prenylation, myristoylation, farnesylation, attachment of a fatty acid, attachment of a GPI anchor, nucleotidylation (e.g., ADP-ribosylation), or the like. As just a few examples, the enzyme can be a protease, protein phosphatase, protein kinase (e.g., a tyrosine kinase or serine/threonine kinase), ubiquitin activating enzyme, ubiquitin protein ligase, glycosyltransferase, ADP-ribosylase (e.g., cholera toxin or pertussis toxin), prenyl transferase (e.g., farnesyl transferase or geranylgeranyltransferase), protein methyltransferase (e.g., histone lysine methyltransferase or histone arginine methyltransferase), or protein acetyltransferase (e.g., histone acetyltransferase or lysine acetyltransferase). The substrate is optionally a specific substrate (acted on only by a single type of catalytic molecule, e.g., under a defined set of reaction conditions), or a generic substrate (acted on by more than one member of a class of catalytic molecules).

The polypeptide substrate optionally comprises a label, e.g., a fluorescent or other label. In one class of embodiments, a signal from the label is sensitive to the state of the substrate. In other words, the signal changes when the enzyme acts on the substrate; the signal from the label is different when the substrate is in the first state, on which the enzyme acts, than when the substrate has been converted to the second state by action of the enzyme. For example, the signal from the label can be a fluorescent emission at a first wavelength whose intensity increases or decreases when the enzyme acts upon the substrate (e.g., decreases by at least about 25%, at least about 50%, at least about 75%, at least about 90%, at least about 95%, or at least about 98%, or increases at least about 2 fold, at least about 5 fold, at least about 10 fold, at least about 20 fold, at least about 50 fold, at least about 100 fold, or even at least about 200 fold). For example, the label can be an environmentally sensitive label whose signal changes when the environment of the label changes as a result of action of the enzyme on the substrate, the label can be a fluorescent label that is quenched when the substrate is in its first state but not quenched when the substrate is in its second state (or vice versa), or the label can be a fluorescent label that exhibits FRET with another fluorophore when the substrate is in its first state but not when the substrate is in its second state (or vice versa). For examples of polypeptide substrates including labels sensitive to the state of the substrate, see, e.g., the Examples section herein below, U.S. patent application publication no. 20040166553 by Nguyen et al. entitled “Caged sensors, regulators and compounds and uses thereof,” U.S. patent application publication no. 20060211075 by Lawrence et al. entitled “Enzyme sensors including environmentally sensitive or fluorescent labels and uses thereof,” and U.S. patent application No. 60/873,753 filed Dec. 6, 2006 by Lawrence et al. entitled “Deeply quenched enzyme sensors and binding sensors,” as well as references herein. In these embodiments, the polypeptide is optionally used as a sensor to assay the activity of the enzyme, e.g., in assays initiated by exposure of the photosensitive polypeptide to light. In other embodiments, the signal from the label is not sensitive to the state of the substrate. In such embodiments the label can be used to normalize results from an enzyme assay employing the polypeptide, e.g., to normalize transfection efficiency in in-cell assays. A variety of labels are known in the art and can be adapted to the practice of the present invention. Further details can be found in the section entitled “Labels” below.

In one exemplary class of embodiments, the enzyme is a protease. A wide variety of polypeptide substrates for various proteases are known in the art and can be adapted to the practice of the present invention. As noted above, the substrate optionally includes a label sensitive to the state of the substrate, e.g., a fluorescent label whose signal changes upon cleavage of the substrate. One such labeled, photosensitive substrate is compound 10, described in the Examples section herein below. Other polypeptide substrates with labels sensitive to cleavage of the substrate have been described in the art, including, for example, protease substrates with fluorogenic leaving groups, substrates with a fluorophore and a quencher on opposite sides of the scissile bond where the fluorophore is unquenched when the substrate is cleaved, and substrates with two fluorophores capable of exhibiting FRET with each other positioned one on either side of the scissile bond. See, e.g., U.S. patent application publication no. 20040166553, Funovics et al. (2003) “Protease sensors for bioimaging” Anal Bioanal Chem 377:956-963, and the serine protease, cysteine protease, and aminopeptidase sensors from Harris et al. (2000) “Rapid and general profiling of protease specificity by using combinatorial fluorogenic substrate libraries” Proc Natl Acad Sci USA 97:7754-7759, Grant et al. (2002) “Development of novel assays for proteolytic enzymes using rhodamine-based fluorogenic substrates” J Biomol Screen 7:531-540, Backes et al. (2000) “Synthesis of positional-scanning libraries of fluorogenic peptide substrates to define the extended substrate specificity of plasmin and thrombin” Nat Biotechnol 18:187-93, and Zimmerman et al. (1977) “Sensitive assays for trypsin, elastase, and chymotrypsin using new fluorogenic substrates” Anal Biochem 78:47-51.

Protease substrates can be protected from cleavage by caging of the scissile peptide bond. Thus, in one class of embodiments, the backbone nitrogen of the polypeptide substrate to which the photolabile group is covalently bonded participates in the amide bond that is cleaved by the protease. The amide of an adjacent residue can be caged, additionally or alternatively, as can other amides whose modification interferes with cleavage of the substrate (e.g., amides that form hydrogen bonds that assist in positioning the substrate in the enzyme's active site or the like).

In another exemplary class of embodiments, the enzyme is a protein kinase, e.g., a tyrosine, serine/threonine, histidine, asp/glu, or arginine kinase. Compound 12, described in the Examples section herein below, is one example of a photosensitive kinase substrate (e.g., for PICA). A large number of other kinases and kinase substrates have been described in the art and can be adapted to the practice of the present invention. See, for example, Pinna and Ruzzene (1996) “How do protein kinases recognize their substrates?” Biochim Biophys Acta 1314:191-225. As noted above, the kinase substrate is optionally labeled, e.g., with a label sensitive to the phosphorylation state of the substrate. See, e.g., U.S. patent application publication nos. 20040166553 and 20060211075 and U.S. patent application No. 60/873,753.

The photosensitive polypeptides can be used in biochemical assays of enzyme activity, to detect enzyme activity inside cells and/or organisms, or the like. Thus, the composition optionally includes a cell lysate or a cell, e.g., a cell comprising the photosensitive polypeptide, a cell comprising the enzyme (endogenously or exogenously expressed), or a cell comprising the enzyme and the photosensitive polypeptide. The polypeptide is optionally associated with a cellular and/or subcellular delivery module such as those described in U.S. patent application publication no. 20040166553 to facilitate introduction of the polypeptide into a cell or a subcellular compartment, for example. The photosensitive polypeptide is optionally bound to a solid support, e.g., at a preselected position in an array of different photosensitive polypeptides or to an identifiable set of particles, or optionally includes an oligonucleotide tag that can hybridize to a complementary oligonucleotide on such an array or set of particles. The composition optionally includes a modulator or potential modulator of the activity of the enzyme (e.g., a known or potential activator or inhibitor).

As noted above, the photolabile group (or groups) inhibits the enzyme from acting on the substrate. The photolabile group can inhibit the enzyme from acting upon the substrate, e.g., by at least about 75%, at least about 90%, at least about 95%, or at least about 98%, as compared to the substrate in the absence of the photolabile group. Preferably, the photolabile group prevents the enzyme from acting upon the substrate. Removal of the photolabile group permits the enzyme to act upon the polypeptide substrate.

Useful site(s) of attachment of photolabile group(s) to a given molecule can be determined by techniques known in the art. For example, amino acid residues central to the activity of a polypeptide substrate (e.g., a residue modified by the enzyme, residues located at a binding interface, or the like) can be identified by routine techniques such as scanning mutagenesis, structural analysis, sequence comparisons, site-directed mutagenesis, or the like. Such residues can then be caged by attachment of a photolabile group to the backbone nitrogen of the residue (or of nearby residues), and the activity of the photosensitive substrate can be assayed to determine the efficacy of caging.

Essentially all of the features noted for the methods above apply to these embodiments as well, as relevant: for example, with respect to type of photolabile group, position of the residue to whose backbone nitrogen the photolabile group is attached in the polypeptide, and/or the like. For example, in one class of embodiments, the photolabile group is a derivative of a 2-nitrobenzyl group, e.g., a 4,5-dimethoxy-2-nitrobenzyl group, a 4-methoxy-2-nitrobenzyl group, a 2-nitrobenzyl group, a 2-nitrophenylethyl group, a 1-(4,5-dimethoxy-2-nitrophenyl)ethyl group, or a nitrodibenzofuranyl group. Similarly, the backbone nitrogen can belong to an amino acid residue that is internal to the polypeptide substrate or that is the N-terminal or C-terminal residue of the polypeptide.

Methods using the photosensitive peptides to assay enzyme activity are also a feature of the invention. Accordingly, another general class of embodiments provides methods of assaying an activity of an enzyme. In the methods, the enzyme and a photosensitive polypeptide are contacted. The photosensitive polypeptide comprises a polypeptide substrate for the enzyme and at least one photolabile group covalently bonded to a backbone nitrogen of the polypeptide substrate, which photolabile group inhibits the enzyme from acting on the substrate. The assay is initiated by exposing the enzyme and the photosensitive polypeptide to light of a first wavelength and thereby removing the photolabile group from the polypeptide substrate, and the activity of the enzyme is assayed.

The photolabile group can inhibit the enzyme from acting upon the substrate, e.g., by at least about 75%, at least about 90%, at least about 95%, or at least about 98%, as compared to the substrate in the absence of the photolabile group. Preferably, the photolabile group prevents the enzyme from acting upon the substrate until the photolabile group is removed. Appropriate wavelengths of light for removing many photolabile groups have been described (e.g., 300-360 nm for 2-nitrobenzyl groups and 350 nm for a nitrodibenzofuranyl group); see, e.g., the Examples section herein below, U.S. Pat. No. 5,998,580, and references herein. Conditions for removing a photolabile group can also be determined or optimized according to techniques well known in the art. Instrumentation and devices for delivering uncaging light are likewise known. For example, well-known and useful light sources include e.g., a lamp, a laser (e.g., a laser optically coupled to a fiber-optic delivery system) or a light-emitting compound. See also U.S. patent application publication no. 20050051706 by Witney et al. entitled “Uncaging devices.”

The enzyme's activity can be assayed by detecting conversion of the substrate to product (i.e., from a first to a second state) by essentially any convenient technique. In one class of embodiments, the polypeptide substrate comprises a label where a signal from the label is sensitive to the state of the substrate, and the methods include detecting the signal from the label. Since the signal is sensitive to the state of the substrate, the amount of substrate converted to product is readily determined.

The assay can be, e.g., qualitative or quantitative. As a few examples, the assay can simply indicate whether the activity is present (e.g., an change in the intensity of a signal from a labeled substrate is detected) or absent (e.g., no signal change is detected), or it can indicate the activity is higher or lower than activity in a corresponding control sample (e.g., the change in intensity is greater or less than that in a control assay or sample, e.g., one that includes a known quantity of enzyme or premodified substrate or the like), or it can be used to determine a number of activity units of the enzyme (an activity unit is typically defined as the amount of enzyme which will catalyze the transformation of 1 micromole of the substrate per minute under standard conditions).

Enzyme assays are well known in art, and additional details can be found, e.g., in the Examples section herein below as well as in U.S. patent application publication nos. 20040166553 and 20060211075 and U.S. patent application No. 60/873,753.

The methods can include contacting the enzyme with a modulator (e.g., an activator or inhibitor) of its activity, or with a potential modulator (e.g., to screen for novel activators or inhibitors). Similarly, the methods can include modulating the activity of at least one other enzyme, e.g., by adding an activator or inhibitor of at least one other enzyme that functions (or potentially functions) in an upstream, downstream, or related signaling or metabolic pathway.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the photosensitive polypeptide comprises introducing the photosensitive polypeptide into a cell that comprises the enzyme (endogenously or exogenously).

Essentially all of the features noted for the embodiments above apply to these embodiments as well, as relevant: for example, with respect to type of photolabile group, position of the residue to whose backbone nitrogen the photolabile group is attached in the polypeptide, exemplary substrates, and/or the like. For example, in one class of embodiments, the photolabile group is a derivative of a 2-nitrobenzyl group, e.g., any of those described herein.

Kits comprising components of compositions of the invention and/or that can be used in practicing the methods of the invention form another feature of the invention. For example, in one class of embodiments, a kit for detecting an activity of an enzyme includes a photosensitive polypeptide and instructions for using the photosensitive polypeptide to detect activity of the enzyme, packaged in one or more containers. The photosensitive polypeptide comprises a polypeptide substrate for the enzyme, a label, wherein a signal from the label is sensitive to the state of the substrate, and at least one photolabile group covalently bonded to a backbone nitrogen of the polypeptide substrate, which photolabile group inhibits (e.g., prevents) the enzyme from acting on the substrate.

The kit optionally also includes one or more buffers, transfection reagents for introducing the photosensitive polypeptide into a cell, controls including a known quantity of the enzyme, and/or the like. Essentially all of the features noted for the embodiments above apply to these embodiments as well, as relevant: for example, with respect to type of photolabile group, position of the residue to whose backbone nitrogen the photolabile group is attached in the polypeptide, exemplary substrates, and/or the like.

### Photosensitive Binding Inhibitors and Uses Thereof

Another aspect of the invention provides caged versions of binding inhibitors that competitively inhibit specific protein-protein interactions. Thus, one general class of embodiments provides a composition that includes a photosensitive polypeptide, which photosensitive polypeptide comprises an inhibitory polypeptide that competes with a first polypeptide for binding to a second polypeptide and at least one photolabile group covalently bonded to a backbone nitrogen of the inhibitory polypeptide. The photolabile group inhibits the inhibitory polypeptide from binding to the second polypeptide, for example, by at least about 75%, at least about 90%, at least about 95%, or at least about 98%, as compared to binding of the inhibitory and second polypeptides in the absence of the photolabile group. Preferably, the photolabile group prevents the inhibitory polypeptide from binding to the second polypeptide. The composition optionally also includes the first polypeptide and/or the second polypeptide.

The first and second polypeptides can be essentially any polypeptides that participate in protein-protein interactions, including, e.g., any of the wide variety of proteins that participate in signal transduction pathways. Typically, in embodiments in which the second polypeptide is an enzyme, the inhibitory polypeptide is not a substrate for the second polypeptide. Similarly, in embodiments in which the second polypeptide is a receptor, the inhibitory polypeptide is typically other than a ligand that activates the receptor.

In one class of embodiments, the inhibitory polypeptide comprises a subsequence of the first polypeptide. In other embodiments, the amino acid sequences of the inhibitory polypeptide and the first polypeptide are unrelated to each other.

The interaction to be inhibited can be an intermolecular or intramolecular interaction. Thus, in certain embodiments, the first polypeptide and the second polypeptide are different molecules, while in other embodiments, the first and second polypeptides are part of the same molecule (i.e., are covalently associated with each other).

The inhibitory polypeptide optionally comprises a label, e.g., a fluorescent or other label. In one class of embodiments, a signal from the label is sensitive to binding of the inhibitory and second polypeptides; thus, a first signal exhibited by the label when the inhibitory polypeptide is not bound to the second polypeptide is distinguishable from a second signal exhibited by the label when the inhibitory polypeptide is bound to the second polypeptide. For example, the signal from the label can be a fluorescent emission at a first wavelength whose intensity increases or decreases when the inhibitory polypeptide binds to the second polypeptide (e.g., decreases by at least about 25%, at least about 50%, at least about 75%, at least about 90%, at least about 95%, or at least about 98%, or increases at least about 2 fold, at least about 5 fold, at least about 10 fold, at least about 20 fold, at least about 50 fold, or at least about 100 fold). For example, the label can be an environmentally sensitive label whose signal changes when the environment of the label changes as a result of binding of the inhibitory polypeptide to the second polypeptide, the label can be a fluorescent label that is quenched when the inhibitory polypeptide binds to the second polypeptide but not quenched when the polypeptides are not bound to each other (or vice versa), or the label can be a fluorescent label that exhibits FRET with another fluorophore when the inhibitory polypeptide is bound to the second polypeptide but not when the polypeptides are not bound to each other (or vice versa). In other embodiments, the signal from the label is not sensitive to binding of the inhibitory and second polypeptides. In such embodiments the label can be used to perform immunolocalization or to normalize results from an assay employing the inhibitory polypeptide, e.g., to normalize transfection efficiency in in-cell assays. A variety of labels are known in the art and can be adapted to the practice of the present invention. Further details can be found in the section entitled “Labels” below.

A wide variety of domains known to recognize various amino acid sequences have been described in the art and can be employed as first or second polypeptides. See, for example, pawsonlab (dot) mshri (dot) on (dot) ca/index (dot) php?option=com_content&task=view&id=30&Itemid=63; a list of phosphopeptide binding domains at folding (dot) cchmc (dot) org/online/SEPdomaindatabase (dot) htm; a list of protein interaction domains at www (dot) mshri (dot) on (dot) ca/pawson/domains (dot) html; a list of protein domains at www (dot) cellsignal (dot) corn/reference/domain/index (dot) asp, which includes consensus binding sites, exemplary peptide ligands, and exemplary binding partners, e.g., for SH-2, 14-3-3, PTB, and WW domains; Kuriyan and Cowburn (1997) “Modular peptide recognition domains in eukaryotic signaling” Annu. Rev. Biophys. Biomol. Struct. 26:259-288; Sharma et al. (2002) “Protein-protein interactions: Lessons learned” Curr. Med. Chem.—Anti-Cancer Agents 2:311-330; Pawson et al. (2001) “SH2 domains, interaction modules and cellular wiring” Trends Cell Biol. 11:504-11; Forman-Kay and Pawson (1999) “Diversity in protein recognition by PTB domains” Curr Opin Struct Biol. 9:690-5; and Fu et al. (2000) “14-3-3 Proteins: Structure, Function, and Regulation” Annual Review of Pharmacology and Toxicology 40:617-647. Exemplary domains useful as or in second polypeptides include, but are not limited to, SH2, SH3, PTB, 14-3-3, LIM, PDZ, WW, and FHA domains.

In one exemplary class of embodiments, the inhibitory polypeptide is a proline rich polypeptide, the second polypeptide comprises an SH3 domain, and the first polypeptide (e.g., another proline rich polypeptide) binds to the SH3 domain. In another class of embodiments, the inhibitory polypeptide comprises a phosphorylated serine residue and the second polypeptide comprises a 14-3-3 domain. In yet another class of embodiments, the inhibitory polypeptide comprises a phosphorylated tyrosine residue, the second polypeptide comprises an SH2 or PTB domain, and the first polypeptide is one that can bind to the SH2 or PTB domain (e.g., a tyrosine kinase).

In one exemplary class of embodiments, the inhibitory polypeptide comprises amino acid sequence Y⁰X⁺¹X⁺²X⁺³ (SEQ ID NO:9), where X⁺¹ and X⁺² are independently selected from the group consisting of an amino acid residue and an amino acid residue comprising a label, and where X⁺³ is selected from the group consisting of A, V, I, L, M, F, Y, W, and an amino acid residue comprising a label. (Inclusion of a label in the polypeptide is optional.) Examples of such photosensitive inhibitory polypeptides include, but are not limited to, compounds 4 and 5 described in the Examples section herein. In this class of embodiments, optionally the second polypeptide is an SH2 domain (e.g., an Lck SH2 domain), Y⁰ is phosphorylated, and the photolabile group inhibits binding of the photosensitive inhibitory polypeptide to the SH2 domain. The first polypeptide is one that can bind to the SH2 domain, e.g., a tyrosine kinase.

The complex between the inhibitory polypeptide and the second polypeptide optionally has a K_{d} of less than about 100 μM, preferably less than about 5 μM, e.g., less than about 500 nm or less than about 50 nm, and more preferably less than about 1 nm. Similarly, the complex between the first and second polypeptides optionally has a K_{d} of less than about 100 μM, e.g., less than about 5 μM, less than about 500 nm, less than about 50 nm, or even less than about 1 nm.

The photosensitive inhibitory polypeptide can be employed in vitro or in vivo. Thus, the composition optionally includes a cell, for example, a cell comprising the photosensitive inhibitory polypeptide, the first polypeptide, and/or the second polypeptide.

Essentially all of the features noted for the embodiments above apply to these embodiments as well, as relevant: for example, with respect to type of photolabile group, position of the residue to whose backbone nitrogen the photolabile group is attached in the polypeptide, determination of optimal placement of the photolabile group, and/or the like. For example, in one class of embodiments, the photolabile group is a derivative of a 2-nitrobenzyl group, e.g., any of those described herein.

Methods using the photosensitive peptides to inhibit (and optionally monitor) protein-protein interactions are also a feature of the invention. Accordingly, another general class of embodiments provides methods of inhibiting interaction between a first polypeptide and a second polypeptide. In the methods, a photosensitive inhibitory polypeptide is provided that comprises an inhibitory polypeptide that competes with the first polypeptide for binding to the second polypeptide and at least one photolabile group covalently bonded to a backbone nitrogen of the inhibitory polypeptide, which photolabile group inhibits (e.g., prevents) the inhibitory polypeptide from binding to the second polypeptide. The photosensitive inhibitory polypeptide, the first polypeptide, and the second polypeptide are contacted, and the photolabile group is removed from the photosensitive inhibitory polypeptide by exposing the photosensitive inhibitory polypeptide to light of a first wavelength, thereby permitting the inhibitory polypeptide to bind to the second polypeptide in competition with the first polypeptide.

The methods can be used in vitro, e.g., to inhibit interaction of purified or partially purified first and second polypeptides, in a cell lysate, or the like, or they can be used inside cells and/or organisms. Thus, in one class of embodiments, contacting the photosensitive inhibitory polypeptide, the first polypeptide, and the second polypeptide comprises introducing the photosensitive inhibitory polypeptide into a cell comprising the first and second polypeptides (endogenously or exogenously expressed).

In one class of embodiments, the inhibitory polypeptide comprises a label, and the method includes detecting a signal from the label. As for the embodiments above, the signal can be insensitive to binding of the inhibitory and second polypeptides (and used, e.g., for normalization, immunolocalization, etc.), or the signal can be sensitive to binding of the inhibitory and second polypeptides (and used, e.g., to detect such binding). Thus, in certain embodiments, a first signal exhibited by the label when the inhibitory polypeptide is not bound to the second polypeptide is distinguishable from a second signal exhibited by the label when the inhibitory polypeptide is bound to the second polypeptide.

Essentially all of the features noted for the embodiments above apply to these embodiments as well, as relevant: for example, with respect to type of photolabile group, position of the residue to whose backbone nitrogen the photolabile group is attached in the polypeptide, exemplary inhibitory, first, and/or second polypeptides, and/or the like. For example, in one class of embodiments, the photolabile group is a derivative of a 2-nitrobenzyl group, e.g., any of those described herein.

Kits comprising components of compositions of the invention and/or that can be used in practicing the methods of the invention form another feature of the invention. For example, in one class of embodiments, a kit for inhibiting interaction between a first polypeptide and a second polypeptide includes a photosensitive inhibitory polypeptide and instructions for using the photosensitive inhibitory polypeptide to inhibit binding of the first polypeptide to the second polypeptide, packaged in one or more containers. The photosensitive inhibitory polypeptide comprises an inhibitory polypeptide that competes with the first polypeptide for binding to the second polypeptide and at least one photolabile group covalently bonded to a backbone nitrogen of the inhibitory polypeptide, which photolabile group inhibits (e.g., prevents) the inhibitory polypeptide from binding to the second polypeptide.

The kit optionally also includes one or more buffers, transfection reagents for introducing the photosensitive polypeptide into a cell, and/or the like. Essentially all of the features noted for the embodiments above apply to these embodiments as well, as relevant: for example, with respect to type of photolabile group, position of the residue to whose backbone nitrogen the photolabile group is attached in the polypeptide, exemplary inhibitory, first, and/or second polypeptides, and/or the like.

### Photosensitive SH2-Interacting Peptides

A related aspect of the invention provides photosensitive versions of polypeptides that bind SH2 domains. Thus, one class of embodiments provides a composition comprising a photosensitive polypeptide, which photosensitive polypeptide comprises a polypeptide comprising amino acid sequence Y⁰X⁺¹X⁺² X⁺³ (SEQ ID NO:9), where X⁺¹ and X⁺² are independently selected from the group consisting of an amino acid residue and an amino acid residue comprising a label, and where X⁺³ is selected from the group consisting of A, V, I, L, M, F, Y, W, and an amino acid residue comprising a label, and at least one photolabile group covalently bonded to a backbone nitrogen of the polypeptide. Inclusion of a label in the polypeptide is optional. Such polypeptides can be employed to block protein-protein (e.g., protein-SH2 domain) interactions as described above, or in enzyme sensors such as those described in U.S. patent application publication no. 20060211075 or quenched enzyme sensors such as those described in U.S. patent application No. 60/873,753.

In one exemplary class of embodiments, the polypeptide comprises amino acid sequence X⁻⁴X⁻³X⁻²X⁻¹Y⁰X⁻¹Y⁰X⁺¹X⁺²X⁺³X⁺⁴X⁺⁵ (SEQ ID NO:10), where X⁻⁴, X⁻³, and X⁻² are independently selected from the group consisting of D, E, and an amino acid residue comprising a label, where X⁻¹ and X⁺³ are independently selected from the group consisting of: A, V, I, L, M, F, Y, W, and an amino acid residue comprising a label, where X⁺¹, X⁺², X⁺⁴, and X⁺⁵ are independently selected from the group consisting of an amino acid residue and an amino acid residue comprising a label. Typically the polypeptide comprises at least one label, e.g., e.g., an environmentally sensitive or fluorescent label; see U.S. patent application publication no. 20060211075.

The presence of the photolabile group optionally interferes with binding of the photosensitive polypeptide to an SH2 domain. Thus, in one embodiment, Y⁰ is phosphorylated, and the photolabile group inhibits (e.g., prevents) binding of the photosensitive polypeptide to an SH2 domain. Similarly, the photolabile group can inhibit (e.g., prevent) phosphorylation of the polypeptide by a protein kinase or dephosphorylation by a protein phosphatase.

The composition optionally includes an SH2 domain, a protein kinase, a protein phosphatase, a cell (e.g., a cell including the photosensitive polypeptide), and/or the like. Exemplary photosensitive polypeptides include, but are not limited to, compounds 4 and 5 from the Examples section herein.

Essentially all of the features noted for the embodiments above apply to these embodiments as well, as relevant: for example, with respect to type of photolabile group, position of the residue to whose backbone nitrogen the photolabile group is attached in the polypeptide, inclusion of a label, and/or the like. For example, in one class of embodiments, the photolabile group is a derivative of a 2-nitrobenzyl group, e.g., any of those described herein.

### Side Chain Caged Polypeptides

One general class of embodiments provides a composition that includes a photosensitive polypeptide, which photosensitive polypeptide comprises a first residue comprising a secondary amine in which a photolabile group is covalently bonded to a side chain nitrogen. The first residue is optionally an amino acid residue, for example, lysine, ornithine, (L)-2,3-diaminopropionic acid, (L)-2,4-diaminobutyric acid, homolysine, or aminophenylalanine. The polypeptide optionally includes more than one side chain comprising a photolabile group, e.g., as part of a secondary amine.

Essentially all of the features noted for the methods above apply to these embodiments as well, as relevant: for example, with respect to type of photolabile group, position of the residue to whose side chain nitrogen the photolabile group is attached in the polypeptide, inclusion of a label, and/or the like. For example, in one class of embodiments, the photolabile group is a derivative of a 2-nitrobenzyl group, e.g., a 4,5-dimethoxy-2-nitrobenzyl group, a 4-methoxy-2-nitrobenzyl group, a 2-nitrobenzyl group, a 2-nitrophenylethyl group, a 1-(4,5-dimethoxy-2-nitrophenyl)ethyl group, or a nitrodibenzofuranyl group. Similarly, the side chain nitrogen can belong to an amino acid residue that is internal to the polypeptide or that is the N-terminal or C-terminal residue of the polypeptide. The polypeptide can be an enzyme substrate, a binding inhibitor, a ligand, or essentially any other type of polypeptide. The presence of the photolabile group optionally inhibits (e.g., prevents) activity of the polypeptide.

Methods using the photosensitive polypeptides, for example, to assay enzyme activity, to inhibit (and optionally monitor) protein-protein interactions, or the like, are also a feature of the invention, as are kits and systems including the polypeptides.

### Systems

In one aspect, the invention includes systems, e.g., systems used to practice the methods herein and/or comprising the compositions described herein. The system can include, e.g., a fluid handling element, a fluid containing element, a laser for exciting a fluorescent label, a detector for detecting a signal from a label (e.g., fluorescent emissions from a fluorescent label), a source of uncaging energy for uncaging photosensitive polypeptides, and/or a robotic element that moves other components of the system from place to place as needed (e.g., a multiwell plate handling element). For example, in one class of embodiments, a composition of the invention is contained in a microplate reader or like instrument. In another example, in one class of embodiments, a composition of the invention is contained in an automated peptide synthesizer.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element, and/or laser). The computer can also receive data from other components of the system, e.g., from a detector, and can interpret the data (e.g., by correlating a change in signal from the label with an activity of an enzyme or with a protein-protein interaction), provide it to a user in a human readable format, or use that data to initiate further operations, in accordance with any programming by the user.

### Labels

As noted, the various polypeptides of this invention optionally include one or more labels, e.g., optically detectable labels, such as fluorescent or luminescent labels, and/or non-optically detectable labels, such as magnetic labels. A number of fluorescent labels are well known in the art, including but not limited to, quantum dots, hydrophobic fluorophores (e.g., rhodamine and fluorescein), and green fluorescent protein (GFP) and variants thereof (e.g., cyan fluorescent protein and yellow fluorescent protein). Exemplary fluorescent labels include, but are not limited to, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook.

Likewise, a variety of fluorophore/quencher combinations, using e.g., fluorescence resonance energy transfer (FRET)-based quenching, non-FRET based quenching, or wavelength-shifting harvester molecules, are known. Example combinations include cyan fluorescent protein and yellow fluorescent protein, terbium chelate and TRITC (tetrarhodamine isothiocyanate), europium chelates and allophycocyanin, europium cryptate and Allophycocyanin, fluorescein and tetramethylrhodamine, IAEDANS and fluorescein, EDANS and DABCYL, fluorescein and DABCYL, fluorescein and fluorescein, BODIPY FL and BODIPY FL, and fluorescein and QSY 7 dye. Nonfluorescent acceptors such as DABCYL and QSY 7 and QSY 33 dyes have the particular advantage of eliminating background fluorescence resulting from direct (i.e., nonsensitized) acceptor excitation. See, e.g., U.S. Pat. Nos. 5,668,648; 5,707,804; 5,728,528; 5,853,992; and 5,869,255 to Mathies et al. for a description of FRET dyes.

For use of quantum dots as labels for biomolecules, see, e.g., Dubertret et al. (2002) Science 298:1759; Nature Biotechnology (2003) 21:41-46; and Nature Biotechnology (2003) 21:47-51.

Other optically detectable labels can also be used in the invention. For example, gold beads can be used as labels and can be detected using a white light source via resonance light scattering. See, e.g. www (dot) geniconsciences (dot) com. Suitable non-optically detectable labels are also known in the art. For example, magnetic labels can be used in the invention (e.g., 3 nm superparamagnetic colloidal iron oxide as a label and NMR detection; see, e.g., Nature Biotechnology (2002) 20:816-820).

The labels are optionally environmentally sensitive or environmentally insensitive labels. The fluorescence of an environmentally insensitive fluorescent label is typically not significantly affected by the solvent in which the label is located. For example, the signal from an environmentally insensitive fluorescent label is typically not significantly different whether the label is in an aqueous solution, a less polar solvent (e.g., methanol), or a nonpolar solvent (e.g., hexane). In contrast, the signal from an environmentally sensitive label changes when the environment of the label changes. For example, the fluorescence of an environmentally sensitive fluorescent label changes when the hydrophobicity, pH, and/or the like of the label's environment changes (e.g., upon binding of the substrate module with which the label is associated to a detection module, such that the label is transferred from an aqueous environment to a more hydrophobic environment at the binding interface between the modules). Typically, the signal from an environmentally sensitive label is affected by the solvent in which the label is located. For example, the signal from an environmentally sensitive fluorescent label is typically significantly different when the label is in an aqueous solution versus in a less polar solvent (e.g., methanol) versus in a nonpolar solvent (e.g., hexane). Examples of environmentally sensitive fluorophores include, but are not limited to, those described in U.S. patent application Ser. No. 11/366,221 and references therein, including in US patent application publication 20020055133 by Hahn et al. entitled “Labeled peptides, proteins and antibodies and processes and intermediates useful for their preparation.”

Labels can be attached to polypeptides during synthesis or by postsynthetic reactions by techniques established in the art. For example, a fluorescently labeled residue can be incorporated into a polypeptide during chemical synthesis of the polypeptide. Alternatively, fluorescent labels can be added to polypeptides by postsynthetic reactions. Reactive forms of various fluorophores are commercially available e.g., from Molecular Probes, Inc., or can readily be prepared by one of skill in the art and used for incorporation of the labels into desired molecules. A polypeptide substrate optionally comprises one or more residues incorporated to facilitate attachment of the label, e.g., a Dap, Dab, ornithine, lysine, cysteine, or homocysteine residue (or essentially any other chemically reactive natural or unnatural amino acid derivative or residue) to which the label is attached.

Similarly, signals from the labels (e.g., absorption by and/or fluorescent emission from a fluorescent label) can be detected by essentially any method known in the art (e.g., fluorescence spectroscopy, fluorescence microscopy, etc.). Excitation and emission wavelengths for the exemplary fluorophores described above can be found, e.g., in The Handbook—A Guide to Fluorescent Probes and Labeling Technologies, Tenth Edition, available on the interne at probes (dot) invitrogen (dot) corn/handbook, and in the references above. Techniques such as multicolor detection, detection of FRET (including, e.g., time-resolved or TR-FRET), and the like are well known in the art. FRET (Fluorescence Resonance Energy Transfer) is a non-radiative energy transfer phenomenon in which two fluorophores with overlapping emission and excitation spectra, when in sufficiently close proximity, experience energy transfer by a resonance dipole induced dipole interaction. The phenomenon is commonly used to study the binding of analytes such as nucleic acids, proteins and the like. FRET is a distance dependent excited state interaction in which emission of one fluorophore is coupled to the excitation of another which is in proximity (close enough for an observable change in emissions to occur). Some excited fluorophores interact to form excimers, which are excited state dimers that exhibit altered emission spectra (e.g., phospholipid analogs with pyrene sn-2 acyl chains). See, e.g., Haugland (1996) Handbook of Fluorescent Probes and Research Chemicals published by Molecular Probes, Inc., Eugene, Oreg. e.g., at chapter 13.

As another example, fluorescence polarization can be used. Briefly, in the performance of such fluorescent binding assays, a typically small, fluorescently labeled molecule, e.g., a ligand, antigen, etc., having a relatively fast rotational correlation time, is used to bind to a much larger molecule, e.g., a receptor protein, antibody etc., which has a much slower rotational correlation time. The binding of the small labeled molecule to the larger molecule significantly increases the rotational correlation time (decreases the amount of rotation) of the labeled species, namely the labeled complex over that of the free unbound labeled molecule. This has a corresponding effect on the level of polarization that is detectable. Specifically, the labeled complex presents much higher fluorescence polarization than the unbound, labeled molecule. See, e.g., U.S. patent application publication no. 20040166553.

### Molecular Biological Techniques

In practicing the present invention, many conventional techniques in molecular biology, microbiology, and recombinant DNA technology are optionally used (e.g., for making and/or manipulating cells of the invention, e.g., cells expressing particular enzymes or other polypeptides). These techniques are well known, and detailed protocols for numerous such procedures (including, e.g., in vitro amplification of nucleic acids, cloning, mutagenesis, transformation, cellular transduction with nucleic acids, protein expression, and/or the like) are described in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2002; and Current Protocols in Molecular Biology, F. M. Ausubel et al., eds., Current Protocols, a joint venture between Greene Publishing Associates, Inc. and John Wiley & Sons, Inc., (supplemented through 2006)). Other useful references, e.g. for cell isolation and culture include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. A variety of vectors, including expression vectors, have been described and are readily available to one of skill, as are a large number of cells and cell lines suitable for the maintenance and use of such vectors.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Single Step Solid Phase Preparation of Caged Peptides

The following sets forth a series of experiments that demonstrate synthesis of exemplary photosensitive polypeptides.

As noted above, light-activatable (“caged”) compounds allow the investigator to control the activity of bio-reagents, even after introducing such agents into cells. This property is particularly useful for studying the temporal and spatial dynamics of signaling pathways, biochemical cascades driven by protein-protein interactions. Indeed, a number of caged peptide derivatives (used to disrupt or probe protein-protein interactions) have been recently described. See Walker et al. (1998) Proc Natl Acad Sci USA 95:1568-73, Wood et al. (1998) J. Amer. Chem. Soc. 120:7145-7146, Zou et al. (2001) Angew Chem Int Ed Engl 40:3049-51, Rothman, et al. (2002) Org Lett 4:2865-8, and Veldhuyzen et al. (2003) J Am Chem Soc 125:13358-9; for an alternative strategy to side chain modification, see Taniguchi et al. (2006) J. Am. Chem. Soc 128:696-7. The general strategy for the preparation of these peptidic species is based on the side-chain modification (with photo-labile groups) of key residues required for bio-recognition, such as Ser or Tyr for protein kinases or phosphoSer for 14-3-3 domains. Unfortunately, many amino acids lack side chain functionality necessary for modification, and the preparation of caged derivatives of those that can be modified is typically a multi-step process. A single-step caging strategy is described herein, which is readily implemented as part of the solid phase synthesis protocol and which is even amenable to residues lacking a reactive side chain functional group.

Protein-protein interactions are often dependent upon one or a few key amino acid residues. These residues must be able to achieve the requisite contacts with the protein-binding partner in order for recognition and/or catalysis to occur. In many instances, the amide NH of the essential and/or adjacent residue is crucial for proper orientation of the key side chain. Thus, replacement of the NH hydrogen bond donor with a sterically demanding (photolabile) moiety can significantly compromise recognition or catalysis. This notion has been examined using three different protein interaction domains.

### Photosensitive SH2-Interacting Polypeptide

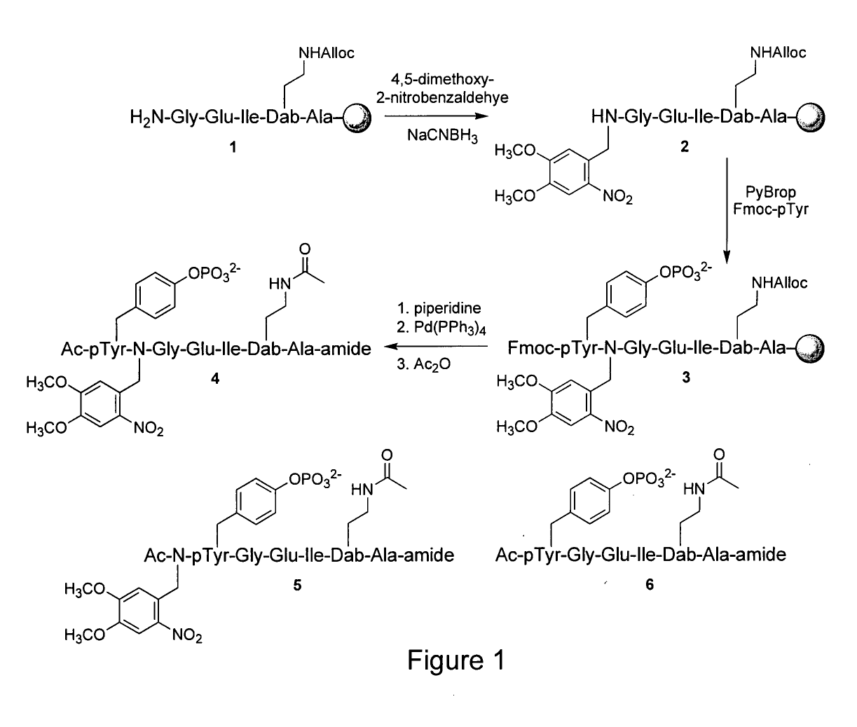

The SH2 domain recognizes and binds to phosphoTyr-containing residues positioned within an appropriate amino acid sequence context (Bradshaw and Waksman (2002) Adv. Protein Chem. 61:161-210, Eck et al. (1993) Nature 362:87-91). For example, the Lck SH2 domain displays a moderate affinity (K_{D}˜1-5 μM) for peptides of the general form Ac-pTyr-Xaa-Xaa-Ile-amide. Previous structural studies have reported that the amide moiety linking the pTyr-Xaa dyad forms a key hydrogen bond with the SH2 domain (Eck et al., supra; coordinates obtained from the Protein Data Bank (ILCJ)). This critical bond helps to properly fix the peptide conformation about the pTyr moiety (e.g., Eck et al., supra). Caged derivative 4 (FIG. 1) was prepared using an Fmoc solid phase peptide synthesis protocol on the Rink Resin. Upon Fmoc deprotection of the P+1 Gly residue (scheme depicted in FIG. 1), the free N-terminal amine of 1 was reductively alkylated with 4,5-dimethoxy-2-nitrobenzaldehyde. Conditions for the subsequent acylation (with phosphoTyr) of the newly formed secondary amine 2 (FIG. 1) were identified: the activating agent PyBrop (Coste et al. (1990) Tetrahedron Lett. 31:669-72) provided the desired Fmoc-pTyr-derivatized peptide 3. The coupling of additional residues (in this case, an acetyl group, and in the examples described below, amino acids) to the growing peptide chain proceeded uneventfully under standard conditions. The corresponding analogue 5, which contains the photo-labile group on the phosphoTyr amide moiety, was also prepared.

K_{d} values were acquired via competition with a previously described dapoxyl-labeled peptide (compound A; see “Experimental procedures” section below, as well as Wang and Lawrence (2005) J. Amer. Chem. Soc. 127:7684-5). This dapoxyl-labeled peptide exhibits a fluorescence change upon binding to the Lck SH2 domain. As expected, caged derivative 4 has a significantly lower SH2 domain affinity (127±6 μM) than the parent peptide 6 (2.6±0.2 μM). By contrast, the difference in affinity between 5 (43±10 μM) and 6 is somewhat more modest. The caged derivative 4 is converted to the high affinity form 6 upon photolysis. Longer photolysis times generate larger yields of the active peptide 6 (see “Experimental procedures” section below). The latter provides a straightforward means to deliver desired quantities of the active species.

### Photosensitive Chymotrypsin Sensor

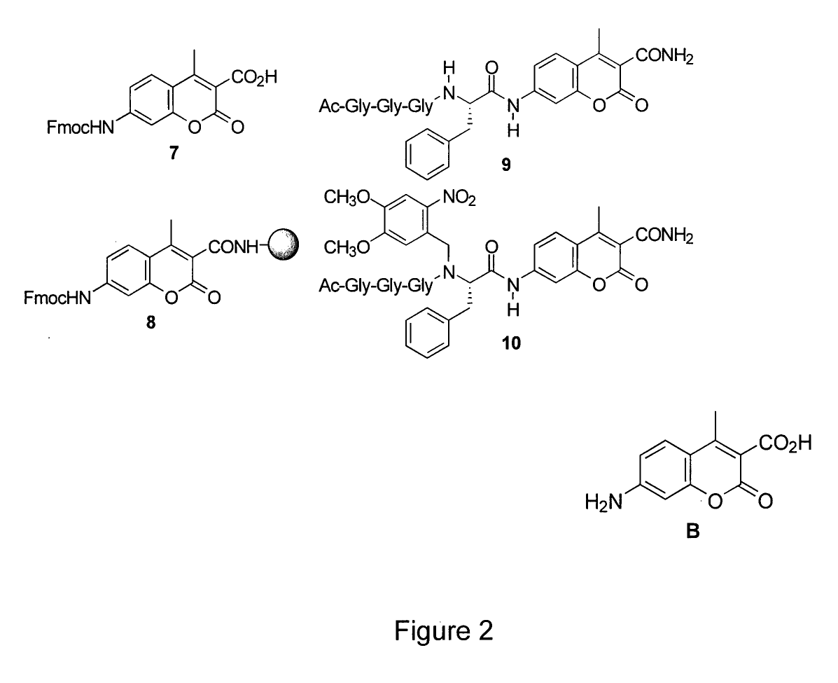

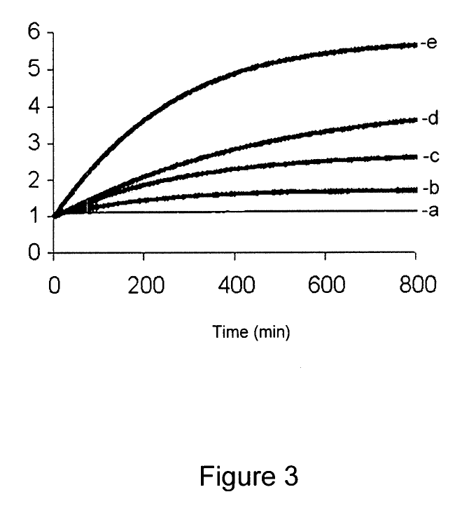

Chymotrypsin substrates are protected from proteolysis via N-methyl substitution of the scissile amide (and at the amide of adjacent residues); Haviv et al. (1993) J. Med. Chem. 36:363-9. N-methylation not only results in the loss of a key hydrogen bond required for substrate recognition, but also introduces a steric factor that compromises active site incorporation. Peptides containing a C-terminal coumarin at the scissile position produce a highly fluorescent coumarin product upon proteolytic cleavage (Zimmerman et al. (1977) Anal. Biochem. 78:47-51 and Backes et al. (2000) Nat. Biotechnol. 18:187-93). In order to render the preparation of these coumarin peptide substrates more amenable to solid phase peptide synthesis, derivative 7 (FIG. 2) was employed. The latter can be directly linked to the resin (8) and then subsequently derivatized using a standard solid phase synthesis Fmoc protocol. Peptide 9, and its corresponding caged analogue 10, were prepared and examined as chymotrypsin substrates. Compound 9 is an efficient substrate (Kₘ=6.3±1.3 μM; Vₘₐₓ=7.2±μmol/min-mg) and exhibits an ˜6-fold enhancement in fluorescence upon complete hydrolysis. By contrast, the caged derivative 10 is essentially inactive as a chymotrypsin substrate (FIG. 3). As in the case of the caged SH2 ligand 4, increasing photolysis times converts increasing amounts of caged analogue 10 to the active substrate 9.

### Photosensitive PKA Substrate

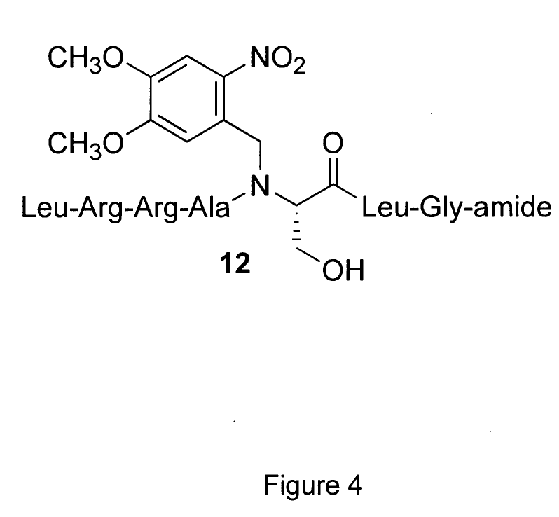

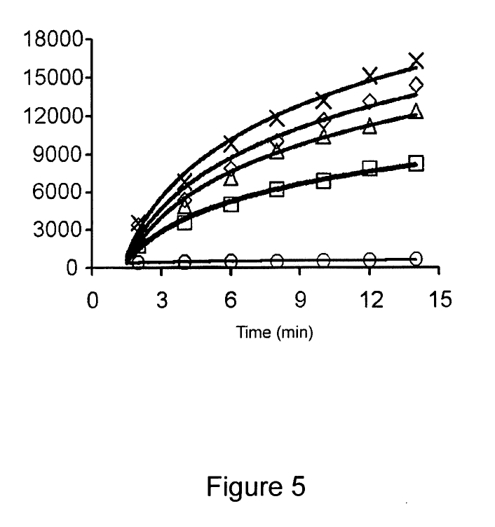

Key hydrogen bonds play a critical role in correctly orienting the phosphorylatable Ser of Leu-Arg-Arg-Ala-Ser-Leu-Gly-amide (11) (SEQ ID NO:6) into the active site of the cAMP-dependent protein kinase (PKA). Indeed, N-methylation of the Ala-Ser amide moiety reduces the PKA-catalyzed efficiency (k_{cat}/Kₘ) of phosphorylation by 7 orders of magnitude (Bramson et al. (1985) J. Biol. Chem. 260:15452-7). Crystallographic studies have revealed that the amide NH of Ser is not only within hydrogen bonding distance of the side chain hydroxyl, but is also oriented toward the incoming phosphoryl group from ATP (Madhusudan et al. (2002) Nature Struc. Biol. 9:273-7; coordinates obtained from the Protein Data Bank (1L3R)). Consequently, it was anticipated that steric bulk positioned at this sensitive site (12) would have a significant deleterious effect on phosphoryl acceptor capability. Compound 12 (FIG. 4) was prepared in a fashion analogous to that outlined in FIG. 1 (i.e., standard Fmoc-based solid phase peptide synthesis interrupted by a single reductive alkylation step). A standard [γ-³³P]ATP assay was employed to assess the efficiency of PKA-catalyzed phosphorylation of peptide 12. In the absence of photolysis, peptide 12 fails to serve as a substrate (FIG. 5). However, photolysis of 12 generates the active substrate 11 (as demonstrated by mass spectrometry) that, upon addition of PKA, furnishes the phosphorylated product (as assessed by scintillation counting). Furthermore, longer photolysis times generate larger quantities of radiolabeled product.

In summary, a single-step, solid phase method has been described herein that furnishes caged peptides in a straightforward fashion. These derivatives, represented by an SH2 ligand and substrates for a protein kinase and a protease, provide a means to control both the timing of target protein interaction as well as the amount of active material unleashed.

### Experimental Procedures

Materials and chemicals were obtained from Fisher and Aldrich, except for piperidine, 1-hydroxybenzotriazole (HOBt), benzotriazole-1-yloxytrispyrrolidinophosphonium hexa-fluorophosphate (PyBop), 5-chloro-1-[bis(dimethylamino)methylene]-1H-benzotriazolium 3-oxide hexafluorophosphate (HCTU), Cl-HOBt, amino acids, and Rink amide resin, which were obtained from Advanced Chemtech, NovaBiochem or Bachem. Dapoxyl sulfonyl chloride was obtained from Molecular Probes. P-81 cellulose unifilter plates were obtained from Whatman. GST-Lck-SH2 and PICA catalytic subunit plasmids were gifts from Dr. Qunzhao Wang and Dr. Hsien-Ming Lee, respectively. Fluorescence assays were performed using a Photon Technology QM-1 spectrofluorimeter, and irradiation experiments utilized an Oriel Mercury Arc Lamp (Model 69907) equipped with a 360 nm colored glass filter (300-400 nm band pass) and an IR filter.

### Synthesis of Peptides 4-6, 9-12, and A

All peptides were synthesized on an Advanced Chemtech Model 90 Tabletop Peptide Synthesizer using a standard 9-fluorenylmethoxycarbonyl (Fmoc) solid-phase peptide synthesis protocol. The side chains of Glu, pTyr, and Ser were protected with t-Bu. The side chain amine of (L)-2,3-diaminobutaric acid (Dab) residue was protected with allyloxycarbonyl (Alloc) group.

(SEQ ID NO: 1)

Ac-pTyr-N(DMNB)Gly-Glu-Ile-Dab(Ac)-Ala-NH₂ (4)

(SEQ ID NO: 2)

Ac-N(DMNB)pTyr-Gly-Glu-Ile-Dab(Ac)-Ala-NH₂ (5)

(SEQ ID NO: 3)

Ac-pTyr-Gly-Glu-Ile-Dab(Ac)-Ala-NH₂ (6)

(SEQ ID NO: 4)

Ac-Gly-Gly-Gly-Phe-coumarin (9)

(SEQ ID NO: 5)

Ac-Gly-Gly-Gly-N(DMNB)Phe-coumarin (10)

(SEQ ID NO: 6)

H₂N-Leu-Arg-Arg-Ala-Ser-Leu-Gly-NH₂ (11)

(SEQ ID NO: 7)

H₂N-Leu-Arg-Arg-Ala-N(DMNB)Ser-Leu-Gly-NH₂ (12)

(SEQ ID NO: 8)

Ac-pTyr-Gly-Glu-Ile-Dab(Dapoxyl)-Ala-NH₂ (A)

Each amino acid (except for the residues discussed in (d) and (e) below) was attached via the following step-by-step protocol:

(a) Deprotection of Fmoc group on the growing peptide chain: 1×15 mL of 30% piperidine in DMF (30 min).
(b) Washing: (i) 3×15 mL of DMF, (ii) 3×15 mL of isopropyl alcohol, (iii) 3×15 mL of CH₂Cl₂.
(c) Three equivalents of the Fmoc-protected amino acid, PyBOP, HOBt hydrate, and six equivalents of N-methylmorpholine in 15 mL of DMF (3 hr).
(d) Coupling of the amino acid immediately after Ile (4, 5, 6, A) was effected via initial exposure to the standard coupling conditions (i.e. with HOBt and PyBop), followed by a subsequent treatment with the amino acid to be coupled in the presence of HOAt and HATU.
(e) Amino acid coupling to the N-DMNB (4,5-dimethoxy-2-nitrobenzyl) substituted residues was achieved via double exposure of the peptide-resin to PyBrop/DIEPA/DMF and six equivalents of the amino acid to be coupled for 6 hr.
Reductive Alkylation: Procedure for the Incorporation of DMNB (Caging Group) onto the Peptide Chain

The N-deprotected peptide-resin was washed with 20 mL of DMF/MeOH/AcOH (9:9:2), drained and then mixed with 3 equiv of 4,5-dimethoxy-2-nitrobenzaldehyde in 10 mL of DMF/MeOH (1:1) for 40 min. The solvent was then removed, and the imine forming reaction repeated. Following solvent removal, the resin was briefly washed with DMF, and then 5 equiv of sodium cyanoborohydride in DMF/MeOH/AcOH (9:9:2) was added and mixed at room temperature for 20 min. The solvent was removed and the resin successively washed with DMF, DMF/H₂O, H₂O, MeOH/CH₂Cl₂, CH₂Cl₂, and DMF. A few mg of resin were cleaved with 97% aqueous TFA and analyzed by ESI and reverse-phase HPLC to ensure the reaction was complete.

### Synthesis of Fmoc-Coumarin Derivative 7

The amine of the commercially available coumarin B (FIG. 2) was protected as the Fmoc derivative using a standard protocol (Backes et al. (2000) Nat. Biotechnol. 18:187-93.) ¹H NMR (DMSO): δ 10.22 (s, 1H), 7.91-7.93 (d, J=7.2 Hz, 2H), 7.73-7.78 (m, 3H), 7.55 (s, 1H), 7.34-7.46 (m, 5H), 4.55-4.57 (d, J=6.6 Hz, 2H), 4.32-4.36 (t, J=6.6 Hz, 1H), 3.58 (s, 2H), 2.36 (s, 3H).

Peptides 9-10 and 12 were prepared using the general Fmoc solid phase peptide synthesis protocol described above.

Peptides 4-6 and A were prepared using the Fmoc protocol described above. Following coupling of all the amino acids, the Dab side chain protected peptide resin was treated with Pd(PPh₃)₄ to selectively remove the allyl protecting group and expose the side chain Dab amine moiety. The peptide was then treated with acetic anhydride (4 equiv) and DIEPA (8 equiv) (4, 5, 6), or dapoxyl sulfonyl chloride (1.3 equiv) and DIPEA (4 equiv) (A), in dry CH₂Cl₂ and allowed to react overnight. Peptide A has been previously described (Wang and Lawrence (2005) J. Amer. Chem. Soc. 127:7684-5).

The peptides were cleaved from the resin (95% TFA, 2.5% triisopropylsilane, 2.5% H₂O), and purified by preparative HPLC (Waters Atlantis dC₁₈ 19×100 mm) using a binary solvent system (solvent A: 0.1% TFA/H₂O; solvent B: 0.1% TFA/CH₃CN) with a ratio of (solvent A):(solvent B) that varied from 97:3 (0 min) to 75:25 (5 min) and then changed in a linear fashion to 65:35 (75 min).

Identities of the purified peptides were confirmed by mass spec.

A: C₄₈H₆₃N₁₀O₁₆P, Mass Calculated m/z 1098.388. found 1097.03 (M−1).
4: C₄₂H₆₀N₉O₁₈P, Mass calculated m/z 1009.379. found 1009.66 (M+).
5: C₄₂H₆₀N₉O₁₈P, Mass calculated m/z: 1009.379. found: 1007.62 (M−2).
6: C₃₃H₅₁N₈O₁₄P, Mass Calculated m/z 814.326. found 812.05 (M−2).
9: C₂₉H₃₂N₆O₈, Mass calculated m/z: 592.23. found: 593.86 (M+1).
10: C₃₈H₄₁N₇O₁₂, Mass calculated m/z 787.28. found 788.36 (M+1).
12: C₄₁H₇₁N₁₅O₁₂, Mass calculated m/z 966.096. found 968.40 (M+2).

### K_{d} Determination of SH2 Domain Sensor Peptide A

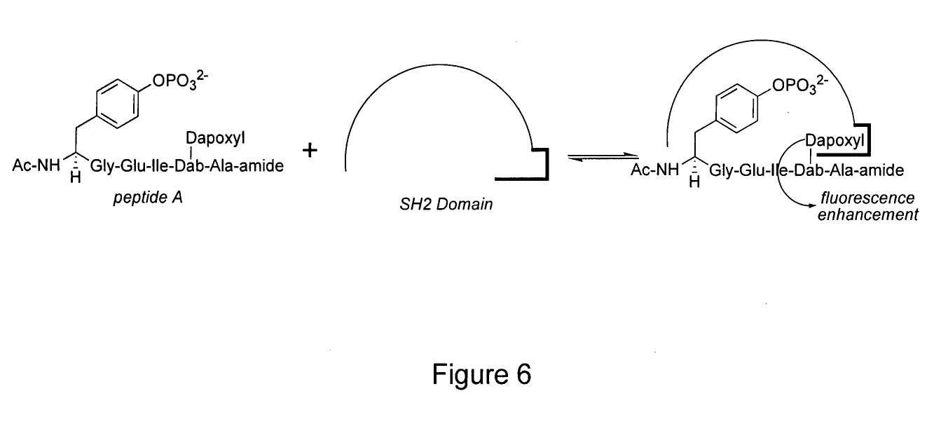

Sensor peptide A exhibits an approximate 10-fold increase in fluorescence upon binding to the Lck SH2 domain, as schematically illustrated in FIG. 6. The K_{d} of the peptide A/Lck SH2 domain complex was determined as follows.

Experiments were conducted with 3 μM of peptide A, 20 μM DTT, varied concentrations of GST-Lck-SH2 (in 10% glycerol) in 33.3 mM of Tris buffer (pH 7.5) in an assay volume of 150 μL. The fluorescence of the solution was monitored on a Photon Technology QM-1 spectrofluorimeter at 30° C. at λₑₓ=395 nm and λₑₘ=535 nm. The fluorescence of the peptide solution was measured with variable concentration of GST-Lck-SH2 (0.1 μM, 0.2 μM, 0.4 μM, 0.8 μM, 1.6 μM, 3.2 μM, 6.4 μM, 12.8 μM, 25.6 μM). Control assays in the absence of GST-Lck-SH2 were also performed at the same concentrations. The K_{d} (1.5±0.3) for the sensor peptide A was determined using the following equations.

$${K}_{d} = \frac{( [ {S}_{T} ] - [ SP ] ) ( [ {P}_{T} ] - [ SP ]}{[ SP ]}$$

$$where \\ [ SP ] = \frac{( {F}_{x} - {F}_{o} )}{( {F}_{max} - {F}_{o} )} [ {S}_{T} ]$$

and [Sₜ]=total [A], Fₓ=fluorescence at specific [Lck-SH2], Fₘₐₓ=maximum fluorescence at excess [Lck-SH2], F₀=starting fluorescence where [Lck-SH2]=0, [Pₜ]=total [Lck-SH2], [SP]=[A/Lck-SH2].

### K_{d} Determination of Caged SH2 Domain Peptides 4 and 5 and Non-Caged Derivative 6

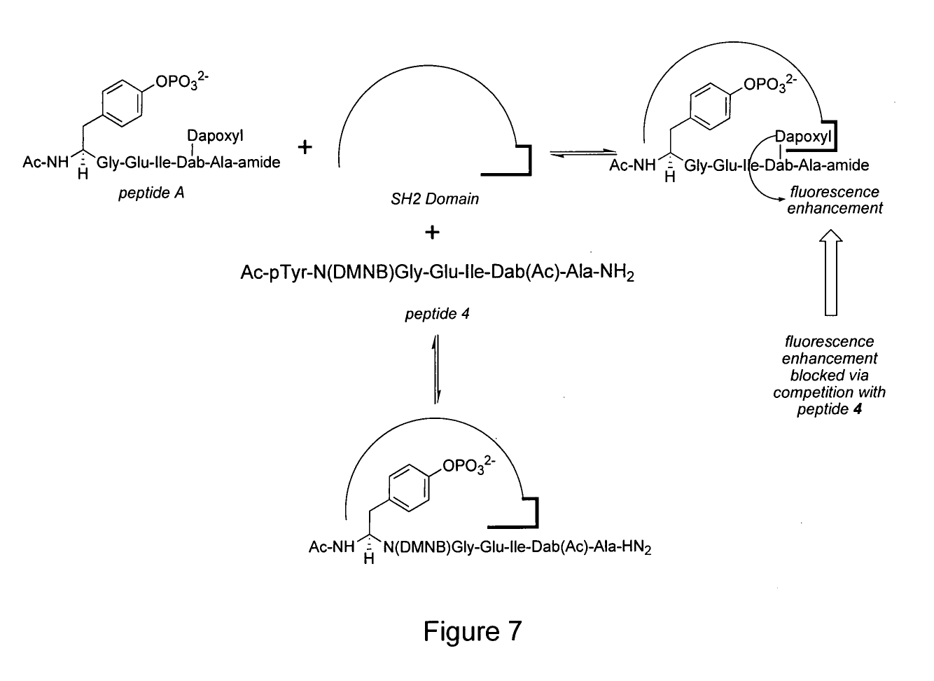

The competition assay schematically illustrated in FIG. 7 was employed to determine K_{d}s for peptides 4, 5, and 6. Experiments were conducted using 3 μM peptide A, 20 μM DTT, 5 μM GST-Lck-SH2 (in 10% glycerol), and 33.3 mM of Tris buffer (pH 7.5) in a total assay volume of 150 μL. The fluorescence of the solution was monitored on a Photon Technology QM-1 spectrofluorimeter at 30° C. at λₑₓ=395 nm and λₑₘ=535 nm. The fluorescence of the peptide A solution was measured in the presence of variable concentrations of peptides 4, 5, or 6. Control assays in the absence of GST-Lck-SH2 were also performed at the same concentrations. The following K_{dx} values were obtained: peptide 4 (127±6 μM), peptide 5 (43±10 μM), and peptide 6 (2.6±0.2).

$${K}_{dx} = \frac{[ P ] ( [ {X}_{T} ] - [ XP ] )}{[ XP ]}$$

$$where \\ [ P ] = \frac{{K}_{d} [ SP ]}{[ {S}_{T} ] - [ SP ]} \\ [ XP ] = [ {P}_{T} ] - [ P ] - [ SP ] \\ [ SP ] = \frac{( {F}_{x} - {F}_{o} )}{( {F}_{max} - {F}_{o} )} [ {S}_{T} ]$$

and [Sₜ]=total [A], Fₓ=fluorescence at a given concentration of peptide X (i.e. 4, 5, or 6), Fₘₐₓ=fluorescence in the absence of peptide X, F₀=fluorescence in the absence of Lck-SH2 domain, K_{d}=binding constant of A/Lck-SH2 domain, [Pₜ]=total [Lck-SH2], [P]=uncomplexed [Lck-SH2], [Xₜ]=total [4, 5, or 6], [PX]=[4, 5, or 6/Lck-SH2 complex], [SP]=[A/Lck-SH2 complex], K_{dx}=dissociation constant of peptide 4, 5, or 6/Lck-SH2 domain complex.

### Photolysis of Caged Peptide 4

Experiments were conducted using 3 μM peptide A, 20 μM DTT, 5 μM GST-Lck-SH2 (in 10% glycerol), peptide 4 (32 μM) in 33.3 mM Tris buffer (pH 7.5) and a total assay volume 150 μL. The fluorescence of the solution was monitored on a Photon Technology QM-1 spectrofluorimeter at 30° C. at λₑₓ=395 nm and λₑₘ=535 nm. Caged peptide (4) was irradiated using an Oriel Mercury Arc Lamp (Model 69907) equipped with a 360 nm colored glass filter (300-400 nm band pass) and an IR filter for various time periods (0-30 min).

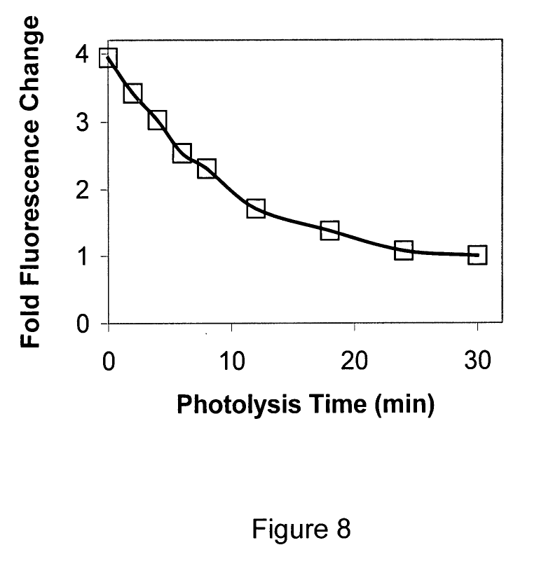

An aliquot from each irradiation time period was added to the assay solution. A decrease in fluorescence intensity is indicative of displacement of the SH2 domain sensor A from the SH2 domain by uncaged peptide (6) (see FIGS. 7 and 8). An analogous set of experiments was performed with peptide 5. The formation of uncaged peptide 6 from caged peptides 4 and 5 was confirmed by analytical HPLC (comparison with the retention time of peptide 6 prepared by solid phase peptide synthesis) and by mass spectrometry.

As shown in FIG. 8, which depicts fluorescence change as a function of photolysis time of caged peptide 4, at t=0 min, the dapoxyl peptide A is completely bound to the Lck-SH2 domain and exhibits a 4-fold enhancement in fluorescence intensity relative to A in the absence of Lck-SH2 domain. Increasing irradiation times converts increasing quantities of 4 to active peptide 6 resulting ultimately in the complete displacement of A from the Lck SH2 domain.

Chymotrypsin Assay and Determination of Kₘ and Vₘₐₓ Values with Peptide 9

Assays were performed in triplicate using peptide 9 concentrations (0.39 μM, 0.78 μM, 1.56 μM, 3.12 μM, 6.25 μM, 12.5 μM, 25 μM) that varied about the Kₘ in 50 mM Tris buffer (pH 8.0), 100 mM NaCl, 5 mM CaCl₂, 10 nM α-chymotrypsin, and 0.01% Tween-20. The peptide 9 stock solution contained 5% DMSO and the assay solution contained less than 1% DMSO. The reaction was monitored on a Photon Technology QM-1 spectrofluorimeter at 30° C. using a λₑₓ=380 nm and a λₑₘ=460 nm.

### Photolysis of Caged-Peptide 10

Assays were conducted using 25 μM light exposed caged peptide 10 in 50 mM Tris buffer (pH 8.0), and 100 mM NaCl, 5 mM CaCl₂, and 0.01% Tween-20 to which α-chymotrypsin was added to initiate the reaction. The fluorescence of the solution was monitored on a Photon Technology QM-1 spectrofluorimeter at 30° C. at λₑₓ=380 nm and λₑₘ=460 nm. Caged peptide 10 was irradiated using an Oriel Mercury Arc Lamp (Model 69907) equipped with a 360 nm colored glass filter (300-400 nm band pass) and an IR filter for various time periods (0, 5, 10, 15, and 20 min).

### Radioactive Assay of the Photolysis Caged PKA Peptide 12

PKA assays were performed in triplicate. 20 μL of a 30 μM solution of 12, which had been photolyzed for different time periods (5, 10, 15, 20, 25, 30 min), was added to each well of 96 multi-well assay plate containing 20 μL assay buffer [100 mM MOPS, 150 mM KCl, 12.5 mM MgCl₂ and 150 μM cold ATP supplemented with 70-163 μCi/well [γ⁻³³P]ATP for radioactive detection]. 10 μL enzyme diluted buffer containing 100 mM MOPS (pH 7.1), 0.125 mg/mL bovine serum albumin, and 8 nM PKA catalytic subunit were added to initiate the reaction. Total reaction volume was 50 μL. After a 12-min incubation time at 30° C., 100 μL of 6% phosphoric acid was added to each well to stop the reaction (total volume: 150 μL). Following an additional 5 min incubation period at ambient temperature, 75 μL from each reaction well was transferred into each well of a Unifilter (P81 cellulose phosphate paper) assay plate. Each well was washed four times with 0.1% phosphoric acid in water. Scintillation solution was added to each well and ³³P-incorporation measured by scintillation counting with a MicroBeta™ TriLux & MicroBeta JET (Perkin Elmer). The formation of the photouncaged peptide was confirmed by ESI mass spectrometry.

Time-Based PKA Assay with Photolyzed Peptide 12

Assays were performed as described above, except that the enzymatic reaction was stopped at different time points (2, 4, 6, 8, 10, 12, 14 min) by adding 6% phosphoric acid.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of making a polypeptide comprising a photolabile group covalently bonded to a backbone nitrogen of a first residue, the method comprising:
during chemical synthesis of the polypeptide, incorporating the first residue into a growing polypeptide, to produce an incorporated first residue; and
reacting an N-terminal amine of the incorporated first residue with at least a first caging compound, to covalently bond the photolabile group to the backbone nitrogen of the first incorporated residue.

2-14. (canceled)

15. A method of making a polypeptide comprising a photolabile group covalently bonded to a side chain nitrogen of a first residue, the method comprising:
during chemical synthesis of the polypeptide, incorporating the first residue into a growing polypeptide, to produce an incorporated first residue; and
reacting a side chain amine of the incorporated first residue with at least a first caging compound, to covalently bond the photolabile group to the side chain nitrogen of the first incorporated residue.

16-20. (canceled)

21. A composition comprising: a photosensitive polypeptide, which photosensitive polypeptide comprises
a) a polypeptide substrate for an enzyme, and
b) at least one photolabile group covalently bonded to a backbone nitrogen of the polypeptide substrate, which photolabile group inhibits the enzyme from acting on the substrate.

22. (canceled)

23. The composition of claim 21, wherein the photolabile group prevents the enzyme from acting upon the substrate.

24. The composition of claim 21, wherein the photolabile group is a derivative of a 2-nitrobenzyl group.

25. The composition of claim 21, wherein the photolabile group has the structure 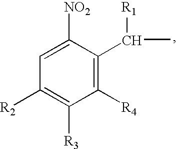
where, independently, R₁ is —H, —CH₃, —CONH₂, or —COO⁻ and R₂, R₃, and R₄ are independently —H, —CH₃, —OCH₃, —CH₂COO⁻, —OH, or —NO₂.

26. The composition of claim 21, wherein the photolabile group is a 4,5-dimethoxy-2-nitrobenzyl group, a 4-methoxy-2-nitrobenzyl group, a 2-nitrobenzyl group, a 2-nitrophenylethyl group, or a 1-(4,5-dimethoxy-2-nitrophenyl)ethyl group.

27. The composition of claim 21, wherein the photolabile group is a derivative of nitrobenzofuran or is a nitrodibenzofuranyl group.

28. The composition of claim 21, wherein the backbone nitrogen belongs to an amino acid residue that is internal to the polypeptide substrate.

29. The composition of claim 21, wherein the polypeptide substrate comprises a label.

30. The composition of claim 29, wherein a signal from the label is sensitive to the state of the substrate.

31. The composition of claim 30, wherein the signal from the label is a fluorescent emission at a first wavelength whose intensity increases or decreases when the enzyme acts upon the substrate.

32. (canceled)

33. The composition of claim 21, wherein the enzyme is a transferase, a hydrolase, a ligase, an isomerase, or a protein phosphatase.

34. The composition of claim 21, wherein the enzyme is a protease.

35. The composition of claim 34, wherein the photosensitive polypeptide has the structure

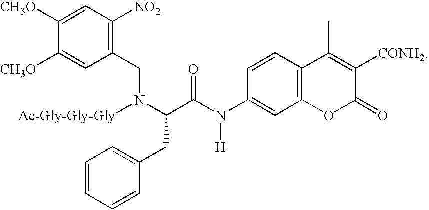

36. The composition of claim 34, wherein the backbone nitrogen of the polypeptide substrate to which the photolabile group is covalently bonded participates in the amide bond that is cleaved by the protease.

37. The composition of claim 21, wherein the enzyme is a protein kinase.

38. The composition of claim 37, wherein the photosensitive polypeptide has the structure

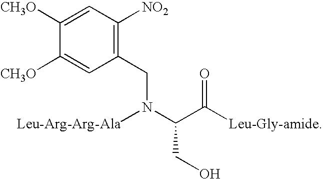

39. (canceled)

40. A method of assaying an activity of an enzyme, the method comprising:
contacting the enzyme and a photosensitive polypeptide, which photosensitive polypeptide comprises
a) a polypeptide substrate for the enzyme, and
b) at least one photolabile group covalently bonded to a backbone nitrogen of the polypeptide substrate, which photolabile group inhibits the enzyme from acting on the substrate;
initiating the assay by exposing the enzyme and the photosensitive polypeptide to light of a first wavelength and thereby removing the photolabile group from the polypeptide substrate; and
assaying the activity of the enzyme.

41-43. (canceled)

44. A kit for detecting an activity of an enzyme, comprising:
a photosensitive polypeptide, which photosensitive polypeptide comprises
a) a polypeptide substrate for the enzyme,
b) a label, wherein a signal from the label is sensitive to the state of the substrate, and
c) at least one photolabile group covalently bonded to a backbone nitrogen of the polypeptide substrate, which photolabile group inhibits the enzyme from acting on the substrate; and
instructions for using the photosensitive polypeptide to detect activity of the enzyme;
packaged in one or more containers.

45. A method of inhibiting interaction between a first polypeptide and a second polypeptide, the method comprising:
a) providing a photosensitive inhibitory polypeptide, which photosensitive inhibitory polypeptide comprises
i) an inhibitory polypeptide that competes with the first polypeptide for binding to the second polypeptide, and
ii) at least one photolabile group covalently bonded to a backbone nitrogen of the inhibitory polypeptide, which photolabile group inhibits the inhibitory polypeptide from binding to the second polypeptide;
b) contacting the photosensitive inhibitory polypeptide, the first polypeptide, and the second polypeptide; and
c) removing the photolabile group from the photosensitive inhibitory polypeptide by exposing the photosensitive inhibitory polypeptide to light of a first wavelength, thereby permitting the inhibitory polypeptide to bind to the second polypeptide in competition with the first polypeptide.

46-55. (canceled)

56. A composition comprising:
a first polypeptide;
a second polypeptide; and
a photosensitive inhibitory polypeptide comprising a) an inhibitory polypeptide that competes with the first polypeptide for binding to the second polypeptide and b) at least one photolabile group covalently bonded to a backbone nitrogen of the inhibitory polypeptide, which photolabile group inhibits the inhibitory polypeptide from binding to the second polypeptide.

57-66. (canceled)

67. A kit for inhibiting interaction between a first polypeptide and a second polypeptide, comprising:
a photosensitive inhibitory polypeptide, which photosensitive inhibitory polypeptide comprises a) an inhibitory polypeptide that competes with the first polypeptide for binding to the second polypeptide and b) at least one photolabile group covalently bonded to a backbone nitrogen of the inhibitory polypeptide, which photolabile group inhibits the inhibitory polypeptide from binding to the second polypeptide; and
instructions for using the photosensitive inhibitory polypeptide to inhibit binding of the first polypeptide to the second polypeptide;
packaged in one or more containers.

68. A composition comprising a photosensitive polypeptide, which photosensitive polypeptide comprises
a) a polypeptide comprising amino acid sequence Y⁰X⁺¹X⁺²X⁺³ (SEQ ID NO:9),
where X⁺¹ and X⁺² are independently selected from the group consisting of an amino acid residue and an amino acid residue comprising a label, and
where X⁺³ is selected from the group consisting of A, V, I, L, M, F, Y, W, and an amino acid residue comprising a label, and
b) at least one photolabile group covalently bonded to a backbone nitrogen of the polypeptide.

69. The composition of claim 68, wherein the polypeptide comprises amino acid sequence X⁻⁴X⁻³X⁻²X⁻¹Y⁰X⁺¹X⁺²X⁺³X⁺⁴X⁺⁵ (SEQ ID NO:10),
where X⁻⁴, X⁻³, and X⁻² are independently selected from the group consisting of D, E, and an amino acid residue comprising a label,
where X⁻¹ and X^{+3 3} are independently selected from the group consisting of: A, V, I, L, M, F, Y, W, and an amino acid residue comprising a label,
where X⁺¹, X⁺², X⁺⁴, and X⁺⁵ are independently selected from the group consisting of an amino acid residue and an amino acid residue comprising a label.

70. The composition of claim 68, wherein Y⁰ is phosphorylated, and wherein the photolabile group inhibits binding of the photosensitive polypeptide to an SH2 domain.

71. The composition of claim 68, wherein the photosensitive polypeptide has the structure

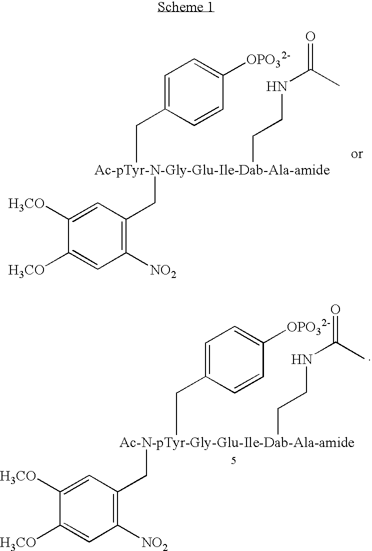

72-74. (canceled)

75. A composition comprising a photosensitive polypeptide, which photosensitive polypeptide comprises a first residue comprising a secondary amine in which a photolabile group is covalently bonded to a side chain nitrogen.

76-79. (canceled)
